(19) United States
(12) Patent Application Publication
(10) Pub. No.: US 2006/0088903 A1
(43) Pub. Date: Apr. 27, 2006

(54) Method for the production of zymosterol and/or the biosynthetic intermediate and/or subsequent products thereof in transgenic organisms

(76) Inventors: Christine Lang, Berlin (DE); Markus Veen, Berlin (DE)

Correspondence Address: LAHIVE & COCKFIELD, 28 STATE STREET, BOSTON, MA, 02109, US

(21) Appl. No.: 10/503,253

(22) PCT Filed: Jan. 22, 2003
(86) PCT No.: PCT/EP03/00590

§ 371 Date: Jul. 29, 2004

(30) Foreign Application Priority Data
Jan. 29, 2002 (DE) 102 03 346.3

(51) Int. Cl. C12P 33/00; A01H 1/00; C12N 1/18; C12N 1/21

(52) U.S. Cl. 435/52; 435/252.3; 800/281; 435/254.2

(57) ABSTRACT

The present invention relates to a method for preparing zymosterol and/or the biosynthetic intermediates and/or secondary products thereof by culturing organisms, in particular yeasts, which, compared to the wild type, have an increased lanosterol-C14-demethylase activity and increased HMG-CoA-reductase activity, to the nucleic acid constructs required for preparing the genetically modified organisms and to the genetically modified organisms themselves, in particular yeasts.

The present invention relates to a method for preparing zymosterol and/or the biosynthetic intermediates and/or secondary products thereof by culturing organisms, in particular yeasts, which, compared to the wild type, have an increased lanosterol C14-demethylase activity and increased HMG-CoA-reductase activity, to the nucleic acid constructs required for preparing the genetically modified organisms and to the genetically modified organisms themselves, in particular yeasts.

Zymosterol, its biosynthetic intermediates of the sterol metabolism, such as, for example, farnesol, geraniol, squalene and lanosterol, and also its biosynthetic secondary products of the sterol metabolism, such as ergosterol (final product of sterol synthesis in yeast and fungi), lathosterol, cholesta-5,7-dienol (provitamin D3) and cholesterol (sterol biosynthesis in mammals) are compounds of high economical value.

The economical importance of ergosterol is based on the one hand on obtaining vitamin D2 from ergosterol via UV irradiation and, on the other hand, on obtaining steroid hormones via biotransformation, starting from ergosterol.

Squalene is used as synthetic building block for the synthesis of terpenes. In hydrogentated form, it is used as squalane in dermatology and cosmetics and also in various derivatives as an ingredient of skincare and haircare products.

Sterols such as zymosterol and lanosterol are also economically usable, with lanosterol being a crude and synthetic pivot for the chemical synthesis of saponins and steroid hormones. Due to its good skin penetration and spreading properties, lanosterol serves as emulsifier and active substance in skin creams.

Cholesta-5,7-dienol (provitamin D3) serves as starting material for the production of vitamin D3 by UV irradiation and is, like cholesterol, the starting material for other steroid hormones.

An economical method for preparing zymosterol and/or the biosynthetic intermediates and/or secondary products thereof is therefore of great importance.

Particularly economical methods are biotechnological methods utilizing natural organisms or organisms optimized by genetic modification, which produce zymosterol and/or the biosynthetic intermediates and/or secondary products thereof.

Most of the genes of the ergosterol metabolism in yeast are known and have been cloned, such as, for example,

nucleic acids encoding an HMG-CoA reductase (HMG) (Bason M. E. et al., (1988) Structural and functional conservation between yeast and human 3-hydroxy-3-methylglutaryl coenzyme A reductases, the rate-limiting enzyme of sterol biosynthesis. Mol Cell Biol 8:3797-3808,

the nucleic acid encoding a truncated HMG-CoA reductase (t-HMG) (Polakowski T, Stahl U, Lang C. (1998) Overexpression of a cytosolic hydroxymethylglutaryl-CoA reductase leads to squalene accumulation in yeast. Appl Microbiol Biotechnol. January; 49(1):66-71,

the nucleic acid encoding a lanosterol C14-demethylase (ERG11) (Kalb V F, Loper J C, Dey C R, Woods C W, Sutter T R (1986) Isolation of a cytochrome P-450 structural gene from Saccharomyces cerevisiae. Gene 45(3):237-45

and the nucleic acid encoding a squalene epoxidase (ERG1) (Jandrositz, A., et al (1991) The gene encoding squalene epoxidase from Saccharomyces cerevisiae: cloning and characterization. Gene 107:155-160.

Furthermore, methods are known which aim at increasing the content of specific intermediates and final products of the sterol metabolism in yeasts and fungi.

EP 486 290 discloses that the content of squalene and other specific sterols such as, for example, zymosterol can be increased in yeasts by increasing the rate of expression of HMG-CoA reductase and, at the same time, interrupting the metabolic pathway of zymosterol-C24-methyl transferase (ERG6) and ergosta-5,7,24(28)-trienol-22dehydrogenase (ERG5).

This method is disadvantageous in that sterols which follow zymosterol in the sterol biosynthesis of yeast are no longer produced.

T. Polakowski, Molekularbiologische Beeinflussung des Ergosterolstoffwechsels der Hefe Saccharomyces cerevisiae, Shaker Verlag Aachen, 1999, pages 59 to 66 discloses that the increase in the rate of expression of HMG-CoA reductase alone, i.e. without interruption of the downstream metabolic flow as in EP 486 290, merely leads to a slight increase in the content of early sterols such as squalene, whereas the content of later sterols such as ergosterol does not substantially change and rather has a tendency to decrease.

WO 99/16886 describes a method for producing ergosterol in yeasts overexpressing a combination of the genes tHMG, ERG9, SAT1 and ERG1.

Tainaka et al., J, Ferment. Bioeng. 1995, 79, 64-66, further describe overexpression of ERG11 (lanosterol-C14-demethylase) leading to accumulation of 4,4-dimethylzymosterol but not ergosterol. The transformant showed, compared to the wild type, an increase in the zymosterol content by a factor of 1.1 to 1.47, depending on fermentation conditions.

It is an object of the present invention to provide a further method for preparing zymosterol and/or the biosynthetic intermediates and/or secondary products thereof having advantageous properties such as an increased product yield.

We have found that this object is achieved by a method for preparing zymosterol and/or the biosynthetic intermediates and/or secondary products thereof by culturing organisms which, compared to the wild type, have an increased lanosterol C14-demethylase activity and an increased HMG-CoA-reductase activity.

Lanosterol-C14-demethylase activity means the enzyme activity of a lanosterol C14-demethylase.

A lanosterol C14-demethylase means a protein having the enzymatic activity to convert lanosterol into 4,4-dimethylcholesta-8,14,24-trienol.

Accordingly, lanosterol-C14-demethylase activity means the amount of lanosterol converted or the amount of 4,4-dimethylcholesta-8,14,24-trienol formed by the protein lanosterol C14-demethylase within a particular time.

In the case of an increased lanosterol-C14-demethylase activity compared to the wild type, therefore, the protein lanosterol C14-demethylase increases the amount of lanosterol converted or the amount of 4,4-dimethylcholesta-8,14,24-trienol formed within a particular time, in comparison with the wild type.

This increase in lanosterol-C14-demethylase activity is preferably at least 5%, further preferably at least 20%, further preferably at least 50%, further preferably at least 100%, more preferably at least 300%, even more preferably at least 500%, in particular at least 600%, of the lanosterol-C14-demethylase activity of the wild type.

HMG-CoA-reductase activity means the enzyme activity of an HMG-COA reductase (3-hydroxy-3-methylglutaryl-coenzyme-A reductase).

A HMG-CoA reductase means a protein having the enzymatic activity to convert 3-hydroxy-3-methylglutaryl-coenzyme-A into mevalonate.

Accordingly, HMG-CoA-reductase activity means the amount of HMG-CoA reductase converted or the amount of 3-hydroxy-3-methylglutaryl coenzyme A formed by the protein HMG-CoA reductase within a particular time.

In the case of an increased HMG-CoA-reductase activity compared to the wild type, therefore, the protein HMG-CoA reductase increases the amount of 3-hydroxy-3-methylglutaryl coenzyme A converted or the amount of mevalonate formed within a particular time, in comparison with the wild type.

This increase in HMG-CoA-reductase activity is preferably at least 5%, further preferably at least 20%, further preferably at least 50%, further preferably at least 100%, more preferably at least 300%, even more preferably at least 500%, in particular at least 600%, of the HMG-CoA-reductase activity of the wild type.

A wild type means the corresponding not genetically unmodified organism.

The increase in lanosterol-C14-demethylase activity, HMG-CoA-reductase activity and in the squalene-epoxidase activity described below may be increased independently of one another in various ways, for example by eliminating inhibiting regulatory mechanisms at the expression and protein levels or by increasing the gene expression of a nucleic acid encoding a lanosterol C14-demethylase, HMG-CoA reductase or squalene epoxidase compared to the wild type, for example by inducing the lanosterol-C14-demethylase gene, HMG-CoA-reductase gene or squalene-epoxidase gene via activators or by introducing one or more nucleic acids encoding a lanosterol C14-demethylase, HMG-CoA reductase or squalene epoxidase into the organism.

According to the invention, increasing the gene expression of a nucleic acid encoding a lanosterol C14-demethylase, an HMG-CoA reductase or a squalene epoxidase also means manipulating the expression of the endogeneous lanosterol C14-demethylases, HMG-CoA reductases or squalene epoxidases intrinsic to the organism, in particular yeasts. This may be achieved, for example, by modifying the promoter DNA sequence of genes coding for lanosterol C14-demethylases, HMG-CoA reductases or squalene-epoxidases. Such a modification which leads to a modified or preferably increased rate of expression of at least one endogenous lanosterol C14-demethylase, HMG-CoA reductase or squalene epoxidase gene may be carried out by deleting or inserting DNA sequences.

As previously described, it is possible to modify expression of at least one endogenous lanosterol C14-demethylase, HMG-CoA reductase or squalene epoxidase by applying exogeneous stimuli. This may be carried out by particular physiological conditions, i.e. by applying foreign substances.

Furthermore, it is possible to achieve a modified or increased expression of at least one endogenous lanosterol C14-demethylase, HMG-CoA reductase or squalene epoxidase gene by the interaction of a regulatory protein which is not present in the untransformed organism with the promoter of said genes.

Such a regulatory protein may be a chimeric protein comprising a DNA-binding domain and a transcriptional activator domain, as described, for example, in WO 96/06166.

In a preferred embodiment, the lanosterol-C14-demethylase activity is increased compared to the wild type by increasing the gene expression of a nucleic acid encoding a lanosterol C14-demethylase.

In another preferred embodiment, gene expression of a nucleic acid encoding a lanosterol C14-demethylase is increased by introducing into the organism one or more nucleic acids encoding a lanosterol C14-demethylase.

For this purpose, it is in principle possible to use any lanosterol-C14-demethylase gene (ERG11), i.e. any nucleic acids encoding a lanosterol C14-demethylase. In the case of genomic lanosterol-C14-demethylase nucleic acid sequences from eukaryotic sources, which contain introns, preferably already processed nucleic acid sequences such as the corresponding cDNAs are to be used, if the host organism is unable or cannot be put into a position to express the corresponding lanosterol C14-demethylase.

Examples of lanosterol-C14-demethylase genes are nucleic acids encoding a lanosterol C14-demethylase from Saccharomyces cerevisiae (Kalb V F, Loper J C, Dey C R, Woods C W, Sutter T R (1986) Isolation of a cytochrome P-450 structural gene from Saccharomyces cerevisiae. Gene 45(3):237-45), Candida albicans (Lamb D C, Kelly D E, Baldwin B C, Gozzo F, Boscott P, Richards W G, Kelly S L (1997) Differential inhibition of Candida albicans CYP51 with azole antifungal stereoisomers. FEMS Microbiol Lett 149(1):25-30), Homo sapiens (Stromstedt M, Rozman D, Waterman M R. (1996) The ubiquitously expressed human CYP51 encodes lanosterol 14 alpha-demethylase, a cytochrome P450 whose expression is regulated by oxysterols. Arch Biochem Biophys May 1, 1996;329(1):73-81c) oder Rattus norvegicus, Aoyama Y, Funae Y, Noshiro M, Horiuchi T, Yoshida Y. (1994) Occurrence of a P450 showing high homology to yeast lanosterol 14-demethylase (P450(14DM)) in the rat liver. Biochem Biophys Res Commun. June 30;201(3):1320-6)

In this preferred embodiment, the transgenic organisms of the invention thus contain, compared to the wild type, at least one further lanosterol-C14-demethylase gene.

The number of lanosterol-C14-demethylase genes in the transgenic organisms of the invention is at least two, preferably more than two, particularly preferably more than three, very particularly preferably more than five.

All nucleic acids mentioned in the description may be, for example, an RNA, DNA or cDNA sequence.

Preference is given to using in the above-described method nucleic acids which encode proteins comprising the amino acid sequence SEQ. ID. NO. 2 or a sequence which is derived from this sequence by substitution, insertion of deletion of amino acids and which is at least 30%, preferably at least 50%, more preferably at least 70%, even more preferably at least 90%, most preferably at least 95%, identical at the amino acid level to the sequence SEQ. ID. NO. 2, which proteins have the enzymic property of a lanosterol C14-demethylase.

The sequence SEQ. ID. NO. 2 is the amino acid sequence of the lanosterol C14-demethylase from Saccharomyces cerevisiae.

Further examples of lanosterol C14-demethylases and lanosterol-C14-demethylase genes can be readily found, for example, in various organisms whose genomic sequence is known via homology comparisons of the amino acid sequences or the corresponding back-translated nucleic acid sequences from data-bases with the SEQ. ID. NO. 2.

Furthermore, other examples of lanosterol C14-demethylases and lanosterol-C14-demethylase genes can be readily found, for example starting from the sequence SEQ. ID. No. 1, in various organisms whose genomic sequence is not known via hybridization and PCR techniques in a manner known per se.

In the description, the term “substitution” means the replacement of one or more amino acids by one or more amino acids. Preference is given to carrying out “conservative” replacements in which the amino acid replaced has a property similar to that of the original amino acid, for example replacing Glu by Asp, Gln by Asn, Val by Ile, Leu by Ile, Ser by Thr.

Deletion is the replacing of an amino acid by a direct bond. Preferred positions for deletions are the polypeptide termini and the junctions between the individual protein domains.

Insertions are insertions of amino acids into the polypeptide chain, with a direct bond formally being replaced by one or more amino acids.

Identity between two proteins means the identity of the amino acids over the in each case entire length of the protein, in particular the identity which is calculated by comparison with the aid of the Lasergene software from DNASTAR, inc. Madison, Wis. (USA) using the Clustal method (Higgins D G, Sharp P M. Fast and sensitive multiple sequence alignments on a microcomputer. Comput Appl. Biosci. April 1989;5(2):151-1), with the parameters set as follows:

Multiple alignment Parameter:

| | |
|---|---|
| Gap penalty | 10 |
| Gap length penalty | 10 |

Pairwise Alignment Parameter:

| | |
|---|---|
| K-tuple | 1 |
| Gap penalty | 3 |
| Window | 5 |
| Diagonals saved | 5 |

Accordingly, a protein which is at least 30% identical at the amino acid level to the sequence SEQ. ID. NO. 2 means a protein which, when comparing its sequence with the sequence SEQ. ID. NO. 2, is at least 30% identical, in particular according to the above programme algorithm using the above set of parameters.

In a further preferred embodiment, nucleic acids which encode proteins comprising the amino acid sequence of Saccharomyces cerevisiae lanosterol C14-demethylase (SEQ. ID. NO. 2) are introduced into organisms.

Suitable nucleic acid sequences can be obtained, for example, by back translating the polypeptide sequence according to the genetic code.

For this purpose, preference is given to those codons which are frequently used according to the organism-specific codon usage. The codon usage can be readily determined on the basis of computer analyses of other known genes of the organisms in question.

If, for example, the protein is to be expressed in yeast, it is often advantageous to use the yeast codon usage for back translation.

In a particularly preferred embodiment, a nucleic acid comprising the sequence SEQ. ID. NO. 1 is introduced into the organism.

The sequence SEQ. ID. NO. 1 is the genomic Saccharomyces cerevisiae DNA (ORF S0001049) which encodes lanosterol C14-demethylase of the sequence SEQ ID NO. 2.

Furthermore, all above-mentioned lanosterol-C14-demethylase genes can be prepared in a manner known per se from the nucleotide building blocks by chemical synthesis, for example by fragment condensation of individual overlapping complementary nucleic acid building blocks of the double helix. The chemical synthesis of oligonucleotides may be carried out, for example, in a manner known per se according to the phosphoramidite method (Voet, Voet, 2^{nd} edition, Wiley Press New York, pages 896-897). The addition of synthetic oligonucleotides and filling-in of gaps with the aid of the Klenow fragment of DNA polymerase and ligation reactions and also general cloning methods are described in Sambrook et al. (1989), Molecular cloning: A laboratory manual, Cold Spring Harbor Laboratory Press.

In a preferred embodiment, the HMG-CoA-reductase activity is increased compared to the wild type by an increase in gene expression of a nucleic acid encoding an HMG-CoA reductase.

In a particularly preferred embodiment of the method of the invention, gene expression of a nucleic acid encoding an HMG-CoA reductase is increased by introducing into the organism a nucleic acid construct comprising a nucleic acid encoding an HMG-CoA reductase whose expression in the organism is subject to a reduced regulation in comparison with the wild type.

A reduced regulation in comparison with the wild type means a reduced regulation and preferably no regulation at the expression or protein level in comparison with the above-defined wild type.

The reduced regulation may be attained preferably by a promoter which is functionally linked to the coding sequence in the nucleic construct and which, in comparison with the wild-type promoter, is subject to a reduced regulation in the organism.

For example, the medium ADH promoter in yeast is subject only to a reduced regulation and is therefore a particularly preferred promoter in the above-described nucleic acid construct.

This promoter fragment of the ADH12s promoter, also referred to as ADH1 hereinbelow, shows newly constitutive expression (Ruohonen L, Penttila M, Keranen S. (1991) Optimization of Bacillus alpha-amylase production by Saccharomyces cerevisiae. Yeast. May-June;7(4):337-462; Lang C, Looman A C. (1995) Efficient expression and secretion of Aspergillus niger RH5344 polygalacturonase in Saccharomyces cerevisiae. Appl Microbiol Biotechnol. December;44(1-2):147-56.), so that the transcriptional regulation is no longer carried out via intermediates of ergosterol biosynthesis.

Further preferred promoters with reduced regulation are constitutive promoters such as, for example, the yeast TEF1 promoter, the yeast GPD promoter or the yeast PGK promoter (Mumberg D, Muller R, Funk M.(1995) Yeast vectors for the controlled expression of heterologous proteins in different genetic backgrounds. Gene. Apr. 14, 1995;156(1):119-22; Chen C Y, Oppermann H, Hitzeman R A. (1984) Homologous versus heterologous gene expression in the yeast, Saccharomyces cerevisiae. Nucleic Acids Res. December 11;12(23):8951-70.).

In a further preferred embodiment, reduced regulation can be attained by using as an HMG-CoA-reductase-encoding nucleic acid a nucleic acid whose expression in the organism is subject to a reduced regulation in comparison with the orthologous nucleic acid intrinsic to said organism.

Particular preference is given to using a nucleic acid encoding only the catalytic region of HMG-CoA reductase (truncated (t-)HMG-CoA-reductase) as an HMG-CoA-reductase-encoding nucleic acid. This nucleic acid (t-HMG) which has been described in EP 486 290 and WO 99/16886 encodes only the catalytically active moiety of the HMG-CoA reductase whose membrane domain which is responsible for regulation at the protein level is missing. Thus, said nucleic acid is subject to a reduced regulation, in particular in yeast, and leads to an increase in gene expression of HMG-CoA reductase.

In a particularly preferred embodiment, nucleic acids are introduced, preferably via the above-described nucleic acid construct, which encode proteins comprising the amino acid sequence SEQ. ID. NO. 4 or a sequence which is derived from this sequence by substitution, insertion or deletion of amino acids and which is at least 30% identical at the amino acid level to the sequence SEQ. ID. NO. 4, which proteins have the enzymic property of an HMG-CoA reductase.

The sequence SEQ. ID. NO. 4 is the amino acid sequence of the truncated HMG-CoA reductase (t-HMG).

Further examples of HMG-CoA reductases and thus also of the t-HMG-CoA reductases reduced to the catalytic region and of the encoding genes can readily be found, for example, in various organisms whose genomic sequence is known by comparing the homologies of the amino acid sequences or of the corresponding back-translated nucleic acid sequences from databases with the sequence SEQ ID. NO. 4.

Further examples of HMG-CoA reductases and thus also of the t-HMG-CoA reductases reduced to the catalytic region and of the encoding genes can furthermore readily be found, for example starting from the sequence SEQ. ID. No. 3, in various organisms whose genomic sequence is not known via hybridization techniques and PCR techniques in a manner known per se.

Particular preference is given to using a nucleic acid comprising the sequence SEQ. ID. NO. 3 as nucleic acid which encodes a truncated HMG-CoA reductase.

In a particularly preferred embodiment, the reduced regulation is achieved by using as an HMG-CoA-reductase-encoding nucleic acid a nucleic acid whose expression in the organism, compared to the orthologous nucleic acid intrinsic to said organism, is subject to a reduced regulation and by using a promoter which, compared to the wild-type promoter, is subject to a reduced regulation in said organism.

Particular preference is given to a method for preparing zymosterol and/or the intermediates and/or secondary products thereof, in which method an organism is used, which in addition to increased lanosterol-C14-demethylase and HMG-CoA-reductase activities, has an increased squalene-epoxidase activity compared to the wild type.

Squalene-epoxidase activity means the enzyme activity of a squalene epoxidase.

A squalene epoxidase means a protein which has the enzymic activity of converting squalene into squalene epoxide.

Accordingly, squalene-epoxidase activity means the amount of squalene converted or the amount of squalene epoxide formed by the protein squalene epoxide over a particular period of time.

Thus, in the case of an increased squalene-epoxidase activity compared to the wild type, the amount of squalene converted or the amount of squalene epoxide formed by the protein squalene epoxidase is increased in comparison with the wild type.

Preferably, this increase in squalene-epoxidase activity is at least 5%, further preferably at least 20%, further preferably at least 50%, further preferably at least 100%, more preferably at least 300%, still more preferably at least 500%, in particular at least 600%, of the squalene-epoxidase activity of the wild type.

As mentioned above, wild type means the corresponding, genetically unmodified organism.

In a preferred embodiment, the squalene-epoxidase activity is increased compared to the wild type by increasing the gene expression of a nucleic acid encoding a squalene epoxidase.

In a further preferred embodiment, gene expression of a nucleic acid encoding a squalene epoxidase is increased by introducing one or more nucleic acids encoding a squalene epoxidase into the organism.

For this purpose, it is in principle possible to use any squalene-epoxidase gene (ERG1), i.e. any nucleic acid encoding a squalene epoxidase. In the case of genomic squalene-epoxidase nucleic acid sequences from eukaryotic sources, which contain introns, already processed nucleic acid sequences such as the corresponding cDNAs are to be used preferably, if the host organism is unable to express or cannot be made to express the corresponding squalene epoxidase.

Examples of nucleic acids encoding a squalene epoxidase are nucleic acids encoding a squalene epoxidase from Saccharomyces cerevisiae (Jandrositz, A., et al (1991) The gene encoding squalene epoxidase from Saccharomyces cerevisiae: cloning and characterization. Gene 107:155-160, from Mus musculus (Kosuga K, Hata S, Osumi T, Sakakibara J, Ono T. (1995) Nucleotide sequence of a cDNA for mouse squalene epoxidase, Biochim Biophys Acta, February 21;1260(3):345-8b), from Rattus norvegicus (Sakakibara J, Watanabe R, Kanai Y, Ono T. (1995) Molecular cloning and expression of rat squalene epoxidase. J Biol Chem Jan 6;270(1):17-20c) and from Homo sapiens (Nakamura Y, Sakakibara J, Izumi T, Shibata A, Ono T. (1996) Transcriptional regulation of squalene epoxidase by sterols and inhibitors in HeLa cells., J. Biol. Chem. Apr. 5, 1996;271(14):8053-6).

Thus, in this preferred embodiment the transgenic organisms of the invention contain, compared to the wild type, at least one further squalene epoxidase.

The number of squalene epoxidase genes in the transgenic organisms of the invention is at least two, preferably more than two, particularly preferably more than three and very particularly preferably more than five.

Preference is given to using in the above-described method nucleic acids, which encode proteins comprising the amino acid sequence SEQ. ID. NO. 6 or a sequence which is derived from this sequence by substitution, insertion or deletion of amino acids and which is at least 30%, preferably at least 50%, more preferably at least 70%, still more preferably at least 90%, most preferably at least 95%, identical at the amino acid level to the sequence SEQ. ID. NO. 6, which proteins have the enzymic property of a squalene epoxidase.

The sequence SEQ. ID. NO. 6 is the amino acid sequence of the squalene epoxidase from Saccharomyces cerevisiae.

Further examples of squalene epoxidases and squalene-epoxidase genes can readily be found, for example, in various organisms whose genomic sequences are known by comparing the homologies of the amino acid sequences or of the corresponding back-translated nucleic acid sequences from databases with the sequence SEQ ID. NO. 6.

Further examples of squalene epoxidases and squalene-epoxidase genes can furthermore readily be found, for example starting from the sequence SEQ. ID. No. 5, in various organisms whose genomic sequence is not known via hybridization techniques and PCR techniques in a manner known per se.

In a further preferred embodiment, nucleic acids are introduced into organisms, which encode proteins comprising the amino acid sequence of the squalene epoxidase from Saccharomyces cerevisiae(SEQ. ID. NO. 6).

Suitable nucleic acid sequences can be obtained, for example, by back translating the polypeptide sequence according to the genetic code.

For this, preference is given to using those codons which are frequently used according to the organism-specific codon usage. Said codon usage can readily be determined on the basis of computer analyses of other known genes of the organisms in question.

If, for example, the protein is to be expressed in yeast, it is often advantageous to use the yeast codon usage for back translation.

In a particularly preferred embodiment, a nucleic acid comprising the sequence SEQ. ID. NO. 5 is introduced into the organism.

The sequence SEQ. ID. NO. 5 is the genomic DNA from Saccharomyces cerevisiae (ORF S0003407), which encodes the squalene epoxidase of the squence SEQ ID NO. 6.

All of the abovementioned squalene-epoxidase genes can furthermore be prepared in a manner known per se by chemical synthesis from the nucleotide building blocks, for example by fragment condensation of individual overlapping, complementary nucleic acid building blocks of the double helix. The chemical synthesis of oligonucleotides may be carried out, for example, according to the phosphoramidite method (Voet, Voet, 2^{nd} edition, Wiley Press New York, pp. 896-897), in a manner known per se. Annealing of synthetic oligonucleotides and filling-in the gaps with the aid of the Klenow fragment of DNA polymerase and ligation reactions and also general cloning methods are described in Sambrook et al. (1989), Molecular cloning: A laboratory manual, Cold Spring Harbor Laboratory Press.

According to the invention, organisms mean, for example, bacteria, in particular bacteria of the genus Bacillus, Escherichia coli, Lactobacillus spec. or Streptomyces spec.,

for example yeasts, in particular yeasts of the genus Saccharomyces cerecisiae, Pichia pastoris or Klyveromyces spec.,

for example fungi, in particular fungi of the genus Aspergillus spec., Penicillium spec. or Dictyostelium spec.

and also, for example, insect cell lines, which are capable, as wild type or owing to previous genetic modification, of producing zymosterol and/or the biosynthetic intermediates and/or secondary products thereof.

Particularly preferred organisms are yeasts, in particular of the species Saccharomyces cerevisiae, in particular the yeast strains Saccharomyces cerevisiae AH22, Saccharomyces cerevisiae GRF, Saccharomyces cerevisiae DBY747 and Saccharomyces cerevisiae BY4741.

As mentioned above, a wild type means the corresponding genetically unmodified organism. In those cases in which the organism or the wild type cannot be classified unambiguously, wild type preferably means a reference organism for the increase in lanosterol-C14-demethylase activity, the increase in HMG-CoA-reductase activity, the increase in squalene-epoxidase activity or the content of zymosterol and/or the biosynthetic intermediates and/or secondary products thereof. This reference organism is preferably the yeast strain Saccharomyces cerevisiae AH22.

Lanosterol-C14-demethylase activity, HMG-CoA-reductase activity and squalene-epoxidase activity of the genetically modified organism of the invention and also of the reference organism are determined under the following conditions:

The activity of HMG-CoA reductase is determined as described in Th. Polakowski, Molekularbiologische Beeinflussung des Ergosterolstoffwechsels der Hefe Saccharomyces cerevisiae [Influencing the Ergosterol Metabolism of the Yeast Saccharomyces cerevisiae by Molecular Biological Means], Shaker-Verlag, Aachen 1999, ISBN 3-8265-6211-9.

According to this, 10⁹ yeast cells of a 48 h culture are harvested by centrifugation (3500×g, 5 min) and washed in 2 ml of buffer I (100 mM potassium phosphate buffer, pH 7.0). The cell pellet is taken up in 500 μl of buffer 1 (cytosolic proteins) or 2 (100 mM potassium phosphate buffer pH 7.0; 1% Triton X-100) (total proteins), and 1 μl of 500 mM PMSF in isopropanol is added. 500 μl of glass beads (d=0.5 mm) are added to the cells and the cells are disrupted by vortexing 5× for one minute each. The liquid between the glass beads is transferred to a new Eppendorf vessel. Cell debris and membrane components are removed by centrifugation (14000×g) for 15 min. The supernatant is transferred to a new Eppendorf vessel and represents the protein fraction.

The activity of HMG-CoA reductase is determined by measuring consumption of NADPH+H⁺ during reduction of 3-hydroxy-3-methylglutaryl-CoA which is added as substrate.

In a 1000 μl assay mixture, 20 μl of yeast protein isolate are combined with 910 μl of buffer I, 50 μl of 0.1 M DTT and 10 μl of 16 mM NADPH+H⁺. The mixture is adjusted to 30° C. and measured in a spectrophotometer at 340 nm for 7.5 min. The decrease in NADPH, which is measured over this period, is the rate of degradation without addition of substrate and is taken into account as background.

Subsequently, substrate (10 μl of 30 mM HMG-CoA) is added, and measurement continues for another 7.5 min. The HMG-CoA-reductase activity is calculated by determining the specific rate of NADPH degradation.

The activity of lanosterol C14-demethylase is determined as described in Omura, T and Sato, R. (1964) The carbon monoxide binding pigment in liver microsomes. J. Biol. Chem. 239, 2370-2378. In this assay, the amount of P450 enzyme as holoenzyme with bound heme can be semi-quantified. The “active” holoenzyme (with heme) can be reduced by CO and only the CO-reduced enzyme has an absorption maximum at 450 nm. Thus the absorption maximum at 450 nm is a measure for lanosterol-C14-demethylase activity.

The activity is determined by diluting a microsomal fraction (4-10 mg/ml protein in 100 mM potassium phosphate buffer) 1:4 so that the protein concentration used in the assay is 2 mg/ml. The assay is carried out directly in a cuvette.

A spatula-tipful of dithionite (S₂O₄Na₂) is added to the microsomes. The base line is recorded in the 380-500 nm region in a spectrophotometer.

Subsequently, approx. 20-30 CO bubbles are passed through the sample. The absorption is then measured over the same region. The absorption level at 450 nm corresponds to the amount of P450 enzyme in the assay mixture.

The activity of squalene epoxidase is determined as described in Leber R, Landl K, Zinser E, Ahorn H, Spok A, Kohliwein S D, Turnowsky F, Daum G. (1998) Dual localization of squalene epoxidase, Erg1p, in yeast reflects a relationship between the endoplasmic reticulum and lipid particles, Mol. Biol. Cell. February 1998;9(2):375-86.

In this method, a total volume of 500 μl contains from 0.35 to 0.7 mg of microsomal protein or from 3.5 to 75 μg of lipid-particle protein in 100 mM Tris-HCl, pH 7.5, 1 mM EDTA, 0.1 mM FAD, 3 mM NADPH, 0.1 mM squalene 2,3-epoxidase cyclase inhibitor U18666A, 32 μM [³H]squalene dispersed in 0.005% Tween 80.

The assay is carried out at 30° C. After 10 min of pretreatment, the reaction is started by adding squalene and stopped after 15, 30 or 45 min by lipid extraction with 3 ml of chloroform/methanol (2:1 vol/vol) and 750 μl of 0.035% MgCl₂.

The lipids are dried under nitrogen and redissolved in 0.5 ml of chloroform/methanol (2:1 vol/vol). For thin layer chromatography, portions are applied to a Silica Gel 60 plate (0.2 mm) and fractionated using chloroform as eluent. The positions containing [³H]2,3-oxidosqualene and [³H]squalene were scraped off and quantified in a scintillation counter.

Biosynthetic zymosterol intermediates mean all compounds which appear as intermediates during zymosterol biosynthesis in the organism used, preferably the compounds mevalonate, farnesyl pyrophosphate, geraniol pyrophosphate, squalene epoxide, 4-dimethylcholesta-8,14,24-trienol, 4,4-dimethylzymosterol, squalene, farnesol, geraniol, lanosterol and zymosterone.

Biosynthetic secondary products of zymosterol mean all compounds which can be derived biosynthetically from zymosterol in the organism used, i.e. for which zymosterol appears as an intermediate. These may be compounds which the organism used produces naturally from zymosterol, such as, for example, 4,4-dimethylzymosterol, 4-methylzymosterol, fecosterol, ergost-7-enol, episterol, ergosta-5,7-dienol, in particular sterols with 5,7-diene structure in yeast and fungi. However, they also mean compounds which can be produced in the organism from zymosterol only by introducing genes and enzyme activities of other organisms for which the starting organism has no orthologous gene.

It is possible, for example, to produce secondary products from zymosterol which are present naturally only in mammals by introducing mammalian genes into yeast:

Introducing, for example, human or murine nucleic acids encoding a human or murine sterol delta-8-delta-7-isomerase and a human or murine delta-5-desaturase into yeast leads to the production of cholesta-7,24-dienol, cholesta-5,7,24-trienol, lathosterol and/or cholesta-5,7-dienol (provitamin D3).

Introducing a further human or murine nucleic acid which encodes a human or murine delta7-reductase enables the yeast to produce cholesterol.

The biosynthetic secondary products of zymosterol therefore mean in particular fecosterol, episterol, ergosta-5,7-dienol, ergosterol, cholesta-7,24-dienol, cholesta-5,7,24-trienol, lathosterol, cholesta-5,7-dienol (provitamin D3) and/or cholesterol.

Preferred biosynthetic secondary products are ergosterol, lathosterol and/or cholesta-5,7-dienol (provitamin D3).

In order to further increase the content of secondary products of zymosterol, it is additionally advantageous to suppress downstream metabolic pathways, i.e. biosynthetic pathways which do not lead to the desired product.

For example, in the case of the above-described preparation of zymosterol-derived mammalian sterols in yeasts, it is advantageous, in addition to increasing according to the invention the activity of lanosterol C14-demethylase and HMG-CoA reductase and, where appropriate, of squalene epoxidase described below, to suppress or to interrupt the natural biosynthetic pathway from zymosterol to ergosterol in yeast, for example by eliminating or deleting ERG6 (C24-methyltransferase) or ERG5 (delta22-reductase) in yeast, preferably by eliminating or deleting ERG6 and ERG5.

The compounds prepared in the method of the invention may be used in biotransformations, chemical reactions and for therapeutic purposes, for example for producing vitamin D₂ from ergosterol via UV irradiation, producing vitamin D₃ from cholesta-5,7-dienol (provitamin D3) via UV irradiation or for producing steroid hormones via biotransformation starting from ergosterol.

In the inventive method for preparing zymosterol and/or the biosynthetic intermediates and/or secondary products thereof the step of culturing the genetically modified organisms, also referred to as transgenic organisms hereinbelow, is preferably followed by harvesting said organisms and isolating zymosterol and/or the biosynthetic intermediates and/or secondary products thereof from said organisms.

The organisms are harvested in a manner known per se and appropriate for the particular organism. Microorganisms such as bacteria, mosses, yeasts and fungi or plant cells which are cultured in liquid media by fermentation may be removed, for example, by centrifugation, decanting or filtration.

Zymosterol and/or the biosynthetic intermediates and/or secondary products thereof are isolated from the harvested biomass together or each compound is harvested separately in a manner known per se, for example by extraction and, where appropriate, further chemical or physical purification processes such as, for example, precipitation methods, crystallography, thermal separation methods such as rectification methods or physical separation methods such as, for example, chromatography.

The transgenic organisms, in particular yeasts, are preferably prepared either by transforming the starting organisms, in particular yeasts, with a nucleic acid construct containing the above-described, lanosterol-C14-demethylase- and HMG-CoA-reductase-encoding nucleic acids which are functionally linked with one or more regulatory signals ensuring transcription and translation in organisms or by combined transformation of 5said starting organisms, in particular yeasts, with at least two nucleic acid constructs, one nucleic acid construct containing the above-described, lanosterol-C14-demethylase-encoding nucleic acids and a second nucleic acid construct containing the above-described HMG-CoA-reductase-encoding nucleic acids and said nucleic acids being functionally linked in each case with one or more regulatory signals ensuring transcription and translation in organisms.

Nucleic acid constructs in which the encoding nucleic acid sequence is functionally linked to one or more regulatory signals ensuring transcription and translation in organisms, in particular in yeasts, are also referred to as expression cassettes hereinbelow.

Examples of nucleic acid constructs containing said expression cassette are vectors and plasmids.

Accordingly, the invention further relates to nucleic acid constructs, in particular nucleic acid constructs functioning as expression cassettes, which comprise lanosterol-C14-demethylase-encoding nucleic acids and HMG-CoA-reductase-encoding nucleic acids which are functionally linked to one or more regulatory signals ensuring transcription and translation in organisms, in particular in yeasts.

In a preferred embodiment, said nucleic acid construct additionally comprises squalene epoxidase-encoding nucleic acids which are functionally linked to one or more regulatory signals ensuring transcription and translation in organisms, in particular in yeasts.

As an alternative, it is also possible to prepare the transgenic organisms of the invention by transformation with a combination of nucleic acid constructs, said combination comprising
a) a first nucleic acid construct comprising nucleic acids encoding a lanosterol C14-demethylase, which are functionally linked to one or more regulatory signals which ensure transcription and translation in organisms and
b) a second nucleic acid construct comprising nucleic acids encoding a HMG-CoA reductase, which are functionally linked to one or more regulatory signals which ensure transcription and translation in organisms.

In a preferred embodiment, the combination comprises
c) yet another, third nucleic acid construct comprising nucleic acids encoding a squalene epoxidase, which are functionally linked to one or more regulatory signals which ensure transcription and translation in organisms.

The regulatory signals preferably contain one or more promoters which ensure transcription and translation in organisms, in particular in yeasts.

The expression cassettes include regulatory signals, i.e. regulatory nucleic acid sequences, which control expression of the coding sequence in the host cell. According to a preferred embodiment, an expression cassette comprises upstream, i.e. at the 5′ end of the coding sequence, a promoter and downstream, i.e. at the 3′ end, a terminator and, where appropriate, further regulatory elements which are operatively linked to the coding sequence for at least one of the above-described genes located in between.

Operative linkage means the sequential arrangement of promoter, coding sequence, terminator and, where appropriate, further regulatory elements in such a way that each of the regulatory elements can properly carry out its function in the expression of the coding sequence.

The preferred nucleic acid constructs, expression cassettes and plasmids for yeasts and fungi and methods for preparing transgenic yeasts and also the transgenic yeasts themselves are described by way of example below.

A suitable promoter of the expression cassette is in principle any promoter which is able to control the expression of foreign genes in organisms, in particular in yeasts.

Preference is given to using in particular a promoter which is subject to reduced regulation in yeast, such as, for example, the medium ADH promoter.

The expression of this promoter fragment of the ADH12s promoter, also referred to as ADH1 hereinbelow, is merely constitutive expression (Ruohonen L, Penttila M, Keranen S. (1991) Optimization of Bacillus alpha-amylase production by Saccharomyces cerevisiae. Yeast. May-June;7(4):337-462; Lang C, Looman A C. (1995) Efficient expression and secretion of Aspergillus niger RH5344 polygalacturonase in Saccharomyces cerevisiae. Appl Microbiol Biotechnol. December;44(1-2):147-56.), so that transcriptional regulation is no longer carried out via intermediates of ergosterol biosynthesis.

Further preferred promoters with reduced regulation are constitutive promoters such as, for example, the yeast TEF1 promoter, the yeast GPD promoter, or the yeast PGK promoter (Mumberg D, Muller R. Funk M. (1995) Yeast vectors for the controlled expression of heterologous proteins in different genetic backgrounds. Gene. Apr. 14, 1995;156(1):119-22; Chen C Y, Oppermann H, Hitzeman R A. (1984) Homologous versus heterologous gene expression in the yeast, Saccharomyces cerevisiae. Nucleic Acids Res. December 11;12(23):8951-70.).

The expression cassette may also contain inducible promoters, in particular a chemically inducible promoter which can be used to control expression of the lanosterol-C14-demethylase gene, the HMG-CoA-reductase gene or the squalene-epoxidase gene in the organism at a particular time.

Promoter of this kind, such as, for example, the yeast CupI promoter (Etcheverry T. (1990) Induced expression using yeast copper metallothionein promoter. Methods Enzymol. 1990;185:319-29.), the yeast Gall-10 promoter (Ronicke V, Graulich W, Mumberg D, Muller R, Funk M. (1997) Use of conditional promoters for expression of heterologous proteins in Saccharomyces cerevisiae, Methods Enzymol.283:313-22) or the yeast Pho5 promoter (Bajwa W, Rudolph H, Hinnen A.(1987) PHO5 upstream sequences confer phosphate control on the constitutive PHO3 gene. Yeast. March 1987; 3 (1):33-42), may be used, for example.

A suitable terminator of the expression cassette is in principle any terminator which is able to control the expression of foreign genes in organisms, in particular in yeasts.

Preference is given to the tryptophan terminator of yeast (TRP1 terminator).

An expression cassette is preferably prepared by fusing a suitable promoter with the above-described lanosterol-C14-demethylase-, HMG-CoA-reductase- and/or squalene epoxidase-encoding nucleic acids and a terminator according to common recombination and cloning techniques as described, for example, in T. Maniatis, E. F. Fritsch and J. Sambrook, Molecular Cloning: A Laboratory Manual, Cold Spring Harbor Laboratory, Cold Spring Harbor, N.Y. (1989) and in T. J. Silhavy, M. L. Berman and L. W. Enquist, Experiments with Gene Fusions, Cold Spring Harbor Laboratory, Cold Spring Harbor, N.Y. (1984) and in Ausubel, F. M. et al., Current Protocols in Molecular Biology, Greene Publishing Assoc. and Wiley-Interscience (1987).

The nucleic acids of the invention may be prepared synthetically or obtained naturally or may contain a mixture of synthetic and natural nucleic acid components and may also comprise various heterologous gene sections of various organisms.

As described above, preference is given to synthetic nucleotide sequences with codons which are preferred by yeasts. These codons which are preferred by yeasts may be determined from codons which have the highest frequency in proteins and which are expressed in most of the interesting yeast species.

When preparing an expression cassette, it is possible to manipulate various DNA fragments in order to obtain a nucleotide sequence which expediently can be read in the correct direction and is provided with a correct reading frame. The DNA fragments may be linked to one another by attaching adapters or linkers to said fragments.

Expediently, the promoter and terminator regions may be provided in the direction of transcription with a linker or polylinker which contains one or more restriction sites for inserting this sequence. Normally, the linker has from 1 to 10, usually from 1 to 8, preferably from 2 to 6, restriction sites. Generally, the linker is within the regulatory regions less than 100 bp, frequently less than 60 bp, but at least 5 bp, in length. The promoter may be both native or homologous and nonnative or heterologous to the host organism. The expression cassette preferably includes in the 5′-3′ direction of transcription the promoter, a coding nucleic acid sequence or a nucleic acid construct and a region for transcriptional termination. Various termination regions can be exchanged with one another randomly.

It is furthermore possible to use manipulations which provide appropriate restriction cleavage sites or remove excess DNA or restriction cleavage sites. In those cases for which insertions, deletions or substitutions such as, for example, transitions and transversions are suitable, in vitro mutagenesis, primer repair, restriction or ligation can be used.

In suitable manipulations such as, for example, restriction, “chewing-back” or filling-in of protruding ends to form blunt ends, complementary fragment ends may be provided for ligation.

The invention further relates to the use of the above-described nucleic acids, the above-described nucleic acid constructs or the above-described proteins for preparing transgenic organisms, in particular yeasts.

Preferably, said transgenic organisms, in particular yeasts, have an increased content of zymosterol and/or the biosynthetic intermediates and/or secondary products thereof compared to the wild type.

Therefore, the invention further relates to the use of the above-described nucleic acids or the nucleic acid constructs of the invention for increasing the content of zymosterol and/or of the biosynthetic intermediates and/or secondary products thereof in organisms which, as wild type or by genetic manipulation, are capable of producing zymosterol and/or the biosynthetic intermediates and/or secondary products thereof.

The above-described proteins and nucleic acids may be used for producing zymosterol and/or the biosynthetic intermediates and/or secondary products thereof in transgenic organisms.

The transfer of foreign genes into the genome of an organism, in particular of yeast, is referred to as transformation.

For this purpose, methods known per se can be used for transformation, in particular in yeasts.

Examples of suitable methods for transforming yeasts are the LiAC method as described in Schiestl R H, Gietz R D. (1989) High efficiency transformation of intact yeast cells using single stranded nucleic acids as a carrier, Curr Genet. December;16(5-6):339-46, electroporation as described in Manivasakam P, Schiestl R H. (1993) High efficiency transformation of Saccharomyces cerevisiae by electroporation. Nucleic Acids Res. September 11;21(18):4414-5, and preparation of protoplasts, as described in Morgan A J. (1983) Yeast strain improvement by protoplast fusion and transformation, Experientia Suppl. 46:155-66.

The construct to be expressed is preferably cloned into a vector, in particular into plasmids which are suitable for transforming yeasts, such as, for example, the vector systems Yep24 (Naumovski L, Friedberg E C (1982) Molecular cloning of eucaryotic genes required for excision repair of UV-irradiated DNA: isolation and partial characterization of the RAD3 gene of Saccharomyces cerevisiae. J Bacteriol October;152(1):323-31), Yepl3 (Broach J R, Strathern J N, Hicks J B. (1979) Transformation in yeast: development of a hybrid cloning vector and isolation of the CAN1 gene. Gene. 1979 Dec.;8(1):121-33), the pRS series of vectors (centromeric and episomal) (Sikorski R S, Hieter P. (1989) A system of shuttle vectors and yeast host strains designed for efficient manipulation of DNA in Saccharomyces cerevisiae. Genetics. May;122(l):19-27) and the vector systems Ycp19 or pYEXBX.

Accordingly, the invention furthermore relates to vectors, in particular plasmids, which comprise the above-described nucleic acids, nucleic acid constructs or expression cassettes.

The invention further relates to a method for preparing genetically modified organisms by functionally introducing an above-described nucleic acid or an above-described nucleic acid construct into the starting organism.

The invention further relates to said genetically modified organisms, the genetic modification increasing the activity of a lanosterol C14-demethylase and an HMG-CoA reductase, compared to a wild type.

Preferably, lanosterol-C14-demethylase activity is increased, as mentioned above, by increasing, compared to the wild type, gene expression of a nucleic acid encoding a lanosterol C14-demethylase.

Preferably, gene expression of a nucleic acid encoding a lanosterol C14-demethylase is increased compared to the wild type by increasing the copy number of said nucleic acid encoding a lanosterol C14-demethylase in the organism.

Accordingly, the invention preferably relates to an above-described genetically modified organism which contains two or more nucleic acids encoding a lanosterol C14-demethylase.

In a preferred embodiment, HMG-CoA-reductase activity is increased compared to the wild type, as mentioned above, by increasing gene expression of a nucleic acid encoding an HMG-CoA reductase.

In a particularly preferred embodiment of the method of the invention, gene expression of a nucleic acid encoding an HMG-CoA reductase is increased by introducing into the organism a nucleic acid construct comprising a nucleic acid which encodes an HMG-CoA reductase and whose expression in said organism is subject to a reduced regulation, in comparison with the wild type.

Accordingly, the invention relates to an above-described genetically modified organism which contains a nucleic acid construct comprising a nucleic acid which encodes an HMG-CoA reductase and whose expression in said organism, in comparison with the wild type, is subject to a reduced regulation.

In this preferred embodiment, the invention in particular relates to an above-described genetically modified organism in which the nucleic acid construct comprises a promoter which is subject to a reduced regulation in said organism, in comparison with the wild type, and/or in which the HMG-CoA-reductase-encoding nucleic acid used is a nucleic acid which encodes only the catalytic region of HMG-CoA reductase.

Particular preference is given to abovementioned genetically modified organisms in which the genetic modification additionally increases squalene-epoxidase activity compared to a wild type.

Preferably, squalene-epoxidase activity is increased, as mentioned above, by increasing, compared to the wild type, gene expression of a nucleic acid encoding a squalene epoxidase.

Preferably, gene expression of a nucleic acid encoding a squalene epoxidase is increased compared to the wild type by increasing in the organism the copy number of the nucleic acid encoding a squalene epoxidase.

Accordingly, the invention preferably relates to an above-described genetically modified organism which contains two or more nucleic acids encoding a squalene epoxidase.

The above-described genetically modified organisms have, compared to the wild type, an increased content of zymosterol and/or of the biosynthetic intermediates and/or secondary products thereof.

Accordingly, the invention relates to an above-described genetically modified organism which, compared to the wild type, has an increased content of zymosterol and/or of the biosynthetic intermediates and/or secondary products thereof.

Preferred genetically modified organisms are yeasts or fungi which have been genetically modified according to the invention, in particular yeasts which have been genetically modified according to the invention, in particular the yeast species Saccharomyces cerevisiae which has been genetically modified according to the invention, in particular the genetically modified yeast strains Saccharomyces cerevisiae AH22, Saccharomyces cerevisiae GRF, Saccharomyces cerevisiae DBY747 and Saccharomyces cerevisiae BY4741.

In the scope of the present invention, increase of the content of zymosterol and/or the biosynthetic intermediates and/or secondary products thereof preferably means the artificially acquired ability to produce biosynthetically an increased amount of at least one of these compounds mentioned at the beginning in the genetically modified organism compared to the genetically unmodified organism.

Accordingly, as mentioned at the beginning, wild type preferably means the genetically unmodified organism, but in particular the reference organism mentioned at the beginning.

An increased content of zymosterol and/or of the biosynthetic intermediates and/or secondary products thereof in comparison with the wild type means in particular the increase in the content of at least one of the abovementioned compounds in the organism by at least 50%, preferably 100%, more preferably 200%, particularly preferably 400%, in comparison with the wild type.

The content of at least one of the mentioned compounds is preferably determined according to analytical methods known per se and preferably refers to those compartments of the organism in which sterols are produced.

The present invention has the following advantage compared to the state of the art:

The method of the invention makes it possible to increase the content of zymosterol and/or of the biosynthetic intermediates and/or secondary products thereof in the producing organisms, without suppressing the pathway to other secondary products and thus restricting the compound portfolio. During the desired production of specific compounds, suppression or interruption of unwanted metabolic pathways provides an additional increase in the content of the desired product.

The invention is illustrated by the following examples but is not limited to them:

### I. GENERAL EXPERIMENTAL CONDITIONS

1. Restriction
Restriction of the plasmids (1 to 10 μg) was carried out in 30 μl reaction mixtures. For this purpose, the DNA was taken up in 24 μl of H₂O and admixed with 3 μl of the appropriate buffer, 1 ml of BSA (bovine serum albumin) and 2 μl of enzyme. The enzyme concentration was 1 unit/μl or 5 units/μl, depending on the amount of DNA. In some cases, 1 μl of RNase was added to the reaction mixture in order to degrade the tRNA. The reaction mixture was incubated at ₃₇° C. for two hours. The restriction was monitored using a minigel.

2. Gel Electrophoreses
The gel electrophoreses were carried out in minigel or wide minigel apparatuses. The minigels (approx. 20 ml, 8 pockets) and the wide minigels (50 ml, 15 or 30 pockets) consisted of 1% strength agarose in TAE. The running buffer used was 1×TAE. After adding 3 μl of stop solution, the samples (10 μl) were applied. λ-DNA cut with HindIII (bands at: 23.1 kb; 9.4 kb; 6.6 kb; 4.4 kb; 2.3 kb; 2.0 kb; 0.6 kb) served as standard. For fractionation, a voltage of 80 V was applied for 45 to 60 min. Thereafter, the gel was stained in ethidium bromide solution and documented under UV light using the INTAS video documentation system or photographed using an orange filter.

3. Gel Elution
The desired fragments were isolated by means of gel elution. The restriction mixture was applied to several pockets of a minigel and fractionated. Only λ-HindIII and a “sacrifice lane” were stained in ethidium bromide solution, examined under UV light, and the desired fragment was marked. This prevented the DNA of the remaining pockets from being damaged by ethidium bromide and UV light. Putting the stained and unstained gel slices side by side made it possible to excise the desired fragment from the unstained gel slice on the basis of the marking. The agarose slice with the fragment to be isolated was introduced into a dialysis tube, sealed in air-bubble-free together with a small amount of TAE buffer and introduced into the BioRad minigel apparatus. The running buffer was 1× TAE and the voltage was 100 V for 40 min. Afterwards, the polarity was switched for 2 min in order to redissolve DNA sticking to the dialysis tube. The buffer in the dialysis tube, which contained the DNA fragments, was transferred to reaction vessels and subjected to ethanol precipitation. For this purpose, 1/10 volume of 3 M sodium acetate, tRNA (1 μl per 50 μl of solution) and 2.5 volumes of ice-cold 96% strength ethanol were added to the DNA solution. The mixture was incubated at −20° C. for 30 min and then removed by centrifugation at 12000 rpm, 4° C., 30 min. The DNA pellet was dried and taken up in 10 to 50 μl of H₂O (depending on the amount of DNA).

4. Klenow Treatment

The Klenow treatment fills in protruding ends of DNA fragments, resulting in blunt ends. Per 1 μg of DNA, the following reaction mixture was pipetted:

| DNA pellet | + 11 μl | H₂0 |
|---|---|---|
| | + 1.5 μl | 10 × Klenow buffer |
| | + 1 μl | 0.1 M DTT |
| | + 1 μl | nucleotide (dNTP 2 mM) |
| 25 | + 1 μl | Klenow polymerase (1 unit/μl) |

The DNA should be from an ethanol precipitation, in order to prevent contamination from inhibiting the Klenow polymerase. The reaction mixture was incubated at 37° C. for 30 min and the reaction was stopped by incubating for another 5 min at 70° C. The DNA was recovered from the reaction mixture by ethanol precipitation and taken up in 10 μl of H₂O.

5. Ligation
The DNA fragments to be ligated were combined. The final volume of 13.1 μl contained approx. 0.5 μl of DNA with a vector/insert ratio of 1:5. The sample was incubated at 70° C. for 45 seconds, cooled to room temperature (approx. 3 min) and then incubated on ice for 10 min. The ligation buffers were then added: 2.6 μl of 500 mM Tris-HCl pH 7.5 and 1.3 μl of 100 mM MgCl₂, followed by incubation on ice for a further 10 min. After adding 1 μl of 500 mM DTT and 1 μl of 10 mM ATP and another 10 min on ice, 1 μl of ligase (1 unit/pl) was added. The whole treatment should be carried out as free from vibrations as possible so that adjoining DNA ends are not separated again. The ligation was carried out at 14° C. overnight.

6. Transformation of E. coli
Competent Escherichia coli (E. coli) NM522 cells were transformed with the DNA of the ligation mixture. A reaction mixture containing 50 μg of the pScL3 plasmid and a reaction mixture without DNA were run as positive control and zero control, respectively. For each transformation mixture, 100 μl of 8% PEG solution, 10 μl of DNA and 200 μl of competent cells (E. coli NM522) were pipetted into a bench centrifuge tube. The reaction mixtures were put on ice for 30 min and agitated occasionally. Then the heat shock was carried out: 1 min at 42° C. For regeneration, 1 ml of LB medium was added to the cells and the suspension was incubated on a shaker at 37° C. for 90 min. In each case 100 μl of the undiluted reaction mixtures, a 1:10 dilution and 1:100 dilution were plated on LB+ampicillin plates and incubated at 37° C. overnight.

7. Plasmid Isolation from E. coli (Miniprep)
E. coli colonies were grown in 1.5 ml LB +ampicillin medium in bench top centrifuge tubes at 37° C. and 120 rpm overnight. On the next day, the cells were removed by centrifugation at 5000 rpm and 4° C. for 5 min and the pellet was taken up in 50 μl of TE buffer. 100 μl of 0.2 N NaOH, 1% SDS solution were added to and mixed with each reaction mixture, and the mixture was put on ice for 5 min (lysis of the cells). Then, 400 μl of Na acetat/NaCl solution (230 μl of H₂O, 130 μl of 3 M sodium acetate, 40 μl of 5 M NaCl) were added, the reaction mixture was mixed and put on ice for a further 15 min (protein precipitation). After centrifugation at 11000 rpm for 15 minutes, the supernatant containing the plasmid DNA was transferred to an Eppendorf vessel. If the supernatant was not completely clear, centrifugation was repeated. 360 μl of ice-cold isopropanol were added to the supernatant and the reaction mixture was incubated at −20° C. for 30 min (DNA precipitation). The DNA was removed by centrifugation (15 min, 12000 rpm, 4° C.), the supernatant was discarded, the pellet was washed in 100 μl of ice-cold 96% strength ethanol, incubated at −20° C. for 50 min and again removed by centrifugation (15 min, 12000 rpm, 4° C.). The pellet was dried in a Speed Vac and then taken up in 100 μl of H₂O. The plasmid DNA was characterized by restriction analysis. For this purpose, 10 μl of each reaction mixture were restriction-digested and fractionated gel-electrophoretically in a wide minigel (see above).

8. Plasmid Preparation from E. coli (Maxiprep)
In order to isolate large amounts of plasmid DNA, the maxiprep method was carried out. Two flasks with 100 ml of LB+ampicillin medium were inoculated with a colony or with 100 μl of a frozen culture which carries the plasmid to be isolated and incubated at 37° C. and 120 rpm overnight. On the next day, the culture (200 ml) was transferred to a GSA beaker and centrifuged at 4000 rpm (2600×g) for 10 min. The cell pellet was taken up in 6 ml of TE buffer. The cell wall was digested by adding 1.2 ml of lysozyme solution (20 mg/ml of TE buffer) and incubated at room temperature for 10 min. Subsequently, the cells were lysed with 12 ml of 0.2 N NaOH, 1% SDS solution, followed by incubation at room temperature for another 5 min. The proteins were precipitated by adding 9 ml of cooled 3 M sodium acetate solution (pH 4.8) and incubation on ice for 15 minutes. After centrifugation (GSA: 13000 rpm (27500×g), 20 min, 4° C.), the supernatant containing the DNA was transferred to a new GSA beaker and the DNA was precipitated with 15 ml of ice-cold isopropanol and incubation at −20° C. for 30 min. The DNA pellet was washed in 5 ml of ice-cold ethanol and dried in air (approx. 30 to 60 min). Thereafter, it was taken up in 1 ml of H₂O. The plasmid was checked by restriction analysis. The concentration was determined by applying dilutions to a minigel. The salt content was reduced by microdialysis (pore size 0.025 μm) for 30 to 60 minutes.

9. Transformation of Yeast
For the transformation of yeast, a preculture of the strain Saccharomyces cerevisiae AH22 was prepared. A flask containing 20 ml of YE medium was inoculated with 100 μl of the frozen culture and incubated at 28° C. and 120 rpm overnight. The main culture was carried out under the same conditions in flasks containing 100 ml of YE medium which was inoculated with 10 μl, 20 μl or 50 μl of the preculture.

9.1 Preparation of Competent Cells
On the next day, the cells in the flasks were counted by means of a Thoma chamber and the flask containing from 3 to 5×10⁷ cells/ml was chosen for the subsequent procedure. The cells were harvested by centrifugation (GSA: 5000 rpm (4000×g) 10 min). The cell pellet was taken up in 10 ml of TE buffer and distributed into two bench top centrifuge tubes (5 ml each). The cells were removed by centrifugation at 6000 rpm for 3 min and then washed twice with in each case 5 ml of TE buffer. The cell pellet was then taken up in 330 μl of lithium acetate buffer per 10⁹ cells, transferred to a sterile 50 ml Erlenmeyer flask and agitated at 28° C. for one hour. As a result, the cells were competent for transformation.

9.2 Transformation
For each transformation mixture, 15 μl of Herring sperm DNA (10 mg/ml), 10 μl of DNA to be transformed (approx. 0.5 μg) and 330 μl of competent cells were pipetted into a bench top centrifuge tube and incubated at 28° C. for 30 min (without agitation). Then, 700 μl of 50% PEG 6000 were added and the suspension was incubated at 28° C. for another hour, without agitation. This was followed by a heat shock at 42° C. for 5 min. 100 μl of the suspension were plated on selection medium (YNB, Difco) in order to select for leucine prototrophy. In the case of selection for G418 resistance, the cells are regenerated after the heat shock (see under 9.3 regeneration phase).

9.3 Regeneration Phase
Since the selection marker is the resistance to G418, the cells need time to express the resistance gene. 4 ml of YE medium were added to the transformation mixtures which were then incubated on the shaker (120 rpm) at 28° C. overnight. On the next day, the cells were removed by centrifugation (6000 rpm, 3 min), taken up in 1 ml of YE medium, and 100 μl or 200 μl thereof were plated on YE+G418 plates. The plates were incubated at 28° C. for several days.

10. Reaction Conditions for the PCR
The reaction conditions for the polymerase chain reaction must be optimized in each individual case and do not apply absolutely to each reaction mixture. Thus it is possible, inter alia, to vary the amount of DNA used, the salt concentrations and the melting temperature. For our task, it proved advantageous to combine in an Eppendorf vessel which was suitable for use in a thermocycler the following substances: 5 μl of Super Buffer, 8 μl of dNTPs (0.625 μM each), 5′ primer, 3′ primer and 0.2 μg template DNA, dissolved in enough water so as to result in a total volume of 50 μl for the PCR reaction mixture, were added to 2 μl (=0.1 U) of Super Taq polymerase. The reaction mixture was briefly centrifuged and overlayed with a drop of oil. Between 37 and 40 cycles were chosen for amplification.

### II. EXAMPLES

### Example 1

Expression of a Truncated HMG-CoA-Reductase in S. cerevisiae GRF

The nucleic acid sequence coding for the expression cassette of ADH-promoter-tHMG-tryptophan-terminator was amplified from the vector YepH2 (Polakowski et al. (1998) Overexpression of a cytosolic hydroxymethylglutaryl-CoA reductase leads to squalene accumulation in yeast. Appl Microbiol Biotechnol. January;49(1):66-71) by PCR using standard methods as mentioned above under the general reaction conditions.

The primers used here are the DNA oligomers AtHT-5′ (forward: tHMGNotF: 5′-CTGCGGCCGCATCATGGACCAATTGGTGAAAACTG-3′; SEQ. ID. NO. 7) and AtHT-3′(reverse: tHMGXhoR: 5′-AACTCGAGAGACACATGGTGCTGTTGTGCTTC-3′; SEQ. ID. No. 8).

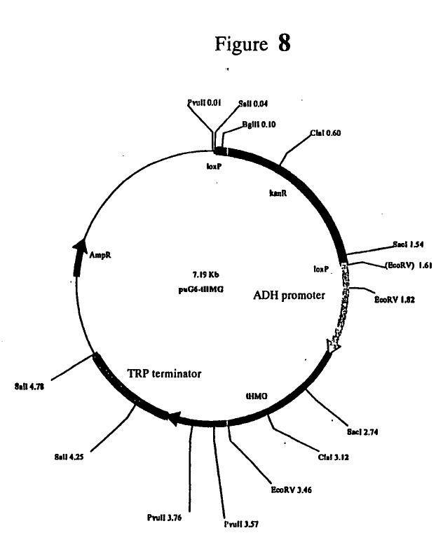

The DNA fragment obtained was cloned, after Klenow treatment into the blunt-ended EcoRV cleavage site of the vector pUG6, resulting in the vector pUG6-tHMG (FIG. 8).

After isolating the plasmid, an extended fragment of the vector pUG-tHMG was amplified by means of PCR so that the resulting fragment consists of the following components: loxP-kanMx-ADH-promoter-tHMG-Ttyptophan-terminator-loxP. The selected primers were oligonucleotide sequences which contain at the 5′ and 3′ protruding ends in each case the 5′ or the 3′ sequence of the URA3 gene and in the annealing region the sequences of the loxP regions 5′ and 3′ of the vector pUG-tHMG. This ensures that on the one hand the entire fragment including KanR and tHMG can be amplified and, on the other hand, said fragment can then be transformed into yeast, and said entire fragment integrates into the yeast URA3 gene locus by homologous recombination.

The resistance to G418 serves as selection marker. The resulting strain S. cerevisiae GRF-tH1ura3 is uracil auxotrophic and contains a copy of the tHMG gene under the control of the ADH promoter and the tryptophan terminator.

In order to subsequently remove the resistance to G418, the resultant yeast strain is transformed with the cre recombinase vector pSH47 (Guldener U, Heck S, Fielder T, Beinhauer J, Hegemann J H. (1996) A new efficient gene disruption cassette for repeated use in budding yeast. Nucleic Acids Res. July 1;24(13):2519-24). Via this vector, cre recombinase is expressed in the yeast leading to elimination of the sequence region within the two loxP sequences by recombination. This results in only one of the two loxP sequences and the ADH-THMG-TRP cassette remaining in the URA3 gene locus. As a consequence, the yeast strain loses the G418 resistance again and is therefore suitable for integrating into or removing from the yeast strain further genes by means of this cre-lox system. The vector pSH47 can then be removed again by a counter selection on YNB agar plates supplemented with uracil (20 mg/L) and FOA (5-fluoroorotic acid) (1 g/L). For this purpose, the cells carrying said plasmid must first be cultured under nonselective conditions and then grown on FOA-containing selective plates. Under these conditions, only cells which are unable to synthesize uracil themselves can grow. They are, in this case, those cells which no longer contain a plasmid (pSH47).

The yeast strain GRF-tH1ura3 and the starting strain GRF were cultured in a culture volume or 20 ml in WMXIII medium at 28° C. and 160 rpm for 48 hours. Subsequently, 500 μl of this preculture were transferred to a 50 ml main culture of the same medium and cultured in a baffled flask at 28° C. and 160 rpm for 4 days.

After 4 days, the sterols were extracted according to the method as described in Parks L W, Bottema C D, Rodriguez R J, Lewis T A. (1985) Yeast sterols: yeast mutants as tools for the study of sterol metabolism. Methods Enzymol. 1985;111:333-46, and analyzed by means of gas chromatography, resulting in the values listed in Table 1. The percentages are based on the yeast dry weight.

TABLE 1

| Sterol content | S. cerevisiae | |
|---|---|---|
| [peak area/gTS] | GRF-tH1ura3 | S. cerevisiae GRF |
| Squalene | 9.93 | 0.1 |
| Lanosterol | 0.83 | 0.31 |
| Zymosterol | 1.18 | 1.07 |
| Ergosterol | 4.71 | 4.71 |
| Fecosterol | 1.10 | 0.64 |
| Episterol | 1.04 | 0.72 |
| Dimethylzymosterol | 0.34 | 0.13 |

### Example 2

Expression of ERG1 in S. cerevisiae GRF tH1ura3 (t-HMG+ERG1)

The squalene-epoxidase sequence was obtained by PCR from genomic DNA of Saccharomyces cerevisiae S288C. The primers used here are the DNA oligomers ERG1-5′ (forward: Erg1NotF: 5′-CTGCGGCCGCATCATGTCTGCTGTTAACGT TGC-3′; SEQ. ID. No. 9) and ERG1-3′ (reverse: Erg1XhoR: 5′-TTCTCGAGTTAACCAATCAACTCACCAAAC-3′; SEQ. ID. No. 10).

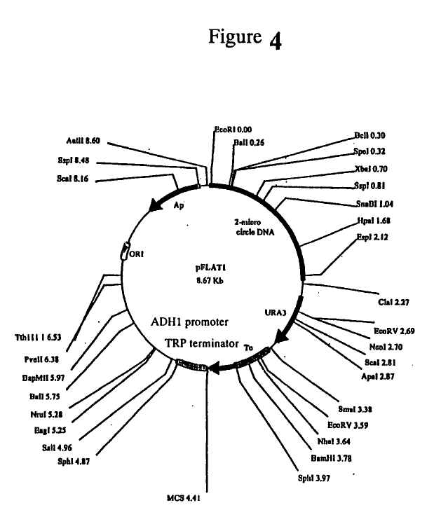

The DNA fragment obtained was treated with restriction enzymes NotI and XhoI and then integrated into the vector pFlat1 (FIG. 4) which had likewise been treated beforehand with the enzymes NotI and XhoI by means of a ligase reaction.

The resulting vector pFlat1-ERG1 (FIG. 5) contains the ERG1 gene under the control of the ADH promoter and the tryptophan terminator.

The expression vector pFlat1-ERG1 was then transformed into the yeast strain S. cerevisiae GRF tH1ura3. The yeast strain S. cerevisiae GRF tH1ura3/pFlat1-ERG1 thus obtained was then cultured in a culture volume of 20 ml in WMXIII medium at 28° C. and 160 rpm for 48 hours. Subsequently, 500 μl of this preculture were transferred to a 50 ml main culture of the same medium and cultured in a baffled flask at 28° C. and 160 rpm for 4 days.

After 4 days, the sterols were extracted according to Example 1 and analyzed by means of gas chromatography, resulting in the values listed in Table 2. The percentages are based on the yeast dry weight.

TABLE 2

| | | S. cerevisiae |
|---|---|---|
| Sterol content | S. cerevisiae | GRF-tH1ura3 |
| [peak area/gTS] | GRF-tH1ura3 | pFlat1-ERG1 |
| Squalene | 9.93 | 3.97 |
| Lanosterol | 0.83 | 3.86 |
| Zymosterol | 1.18 | 1.83 |
| Ergosterol | 4.71 | 4.38 |
| Fecosterol | 1.10 | 1.54 |
| Episterol | 1.04 | 1.09 |
| Dimethylzymosterol | 0.34 | 0.57 |

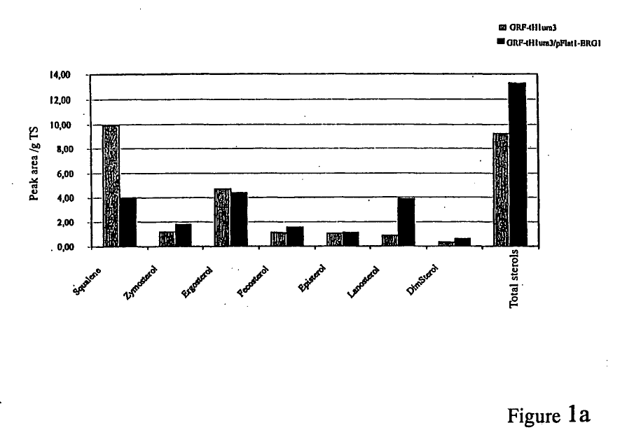

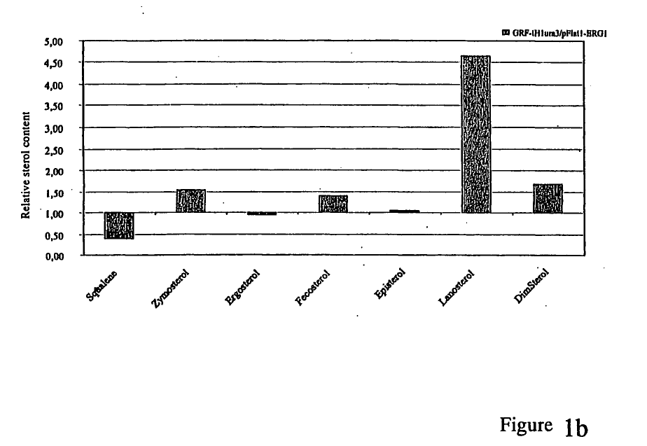

FIGS. 1a and 1b show the absolute increase (1a) and percent increase (1b) in the content of individual sterols in S. cerevisae GRF tH1ura3/pFlat1-ERG1 in comparison with the starting strain S. cerevisae GRF tH1ura3.

### Example 3

Expression of ERG11 in S. cerevisiae GRF-tH1ura3 (t-HMG+ERG11)

The lanosterol C14-demethylase sequence (ERG11) was obtained by PCR from genomic DNA of Saccharomyces cerevisiae S288C.

The primers used here are the DNA oligomers

ERG11-5′ (forward: Erg11NotF:
5′-CTGCGGCCGCAGGATGTCTGCTACCAAGTCAATCG-3′; SEQ. ID. No. 11)
and
ERG11-3′ (reverse: Erg11XhoR:
5′-ATCTCGAGCTTAGATCTTTTGTTCTGGATTTCTC-3′; SEQ ID No. 12).

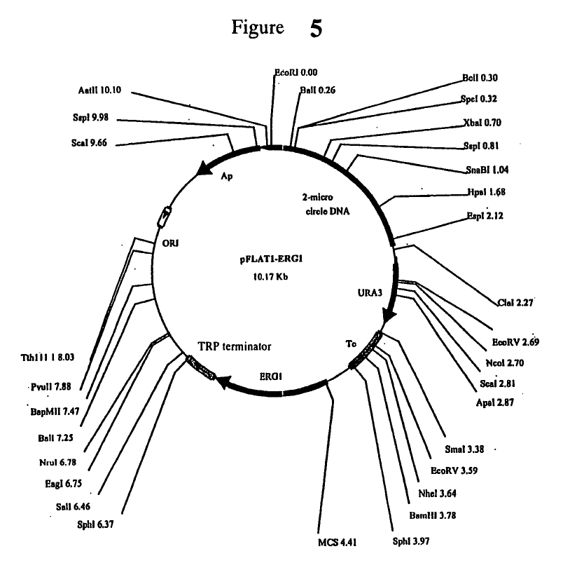

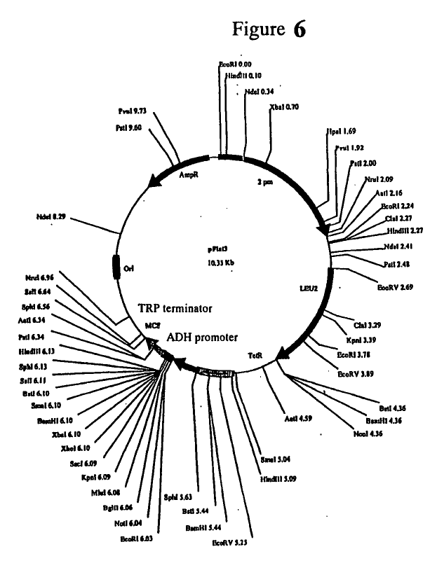

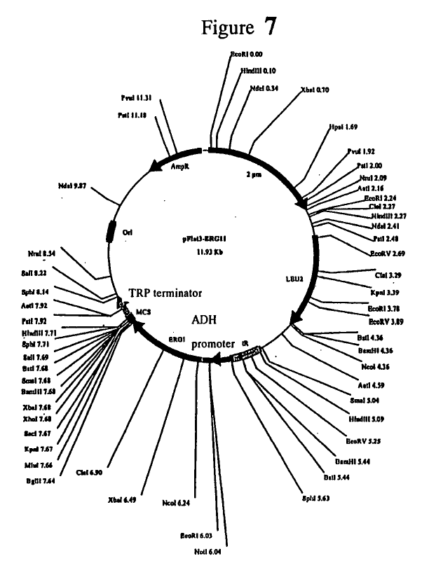

The DNA fragment obtained was treated with restriction enzymes NotI and XhoI and then integrated by means of a ligation reaction into the vector pFlat3 (FIG. 6) which had likewise been treated beforehand with the enzymes NotII and XhoI. The resulting vector pFlat3-ERG11 (FIG. 7) contains the ERG11 gene under the control of the ADH promoter and the tryptophan terminator.

The expression vector pFlat1-ERG11 was then transformed into the yeast strain S. cerevisiae GRF tH1ura3. The yeast strain S. cerevisiae GRF tH1ura3/pFlat1-ERG11 thus obtained was then cultured in a culture volume of 20 ml in WMXIII medium at 28° C. and 160 rpm for 48 hours. Subsequently, 500 μl of this preculture were transferred to a 50 ml main culture of the same medium and cultured in a baffled flask at 28° C. and 160 rpm for 4 days.

After 4 days, the sterols were extracted according to Example 1 and analyzed by means of gas chromatography, resulting in the values listed in Table 3. The percentages are based on the yeast dry weight.

TABLE 3

| Sterol content | S. cerevisiae | S. cerevisiae GRF-tH1ura3/ |
|---|---|---|
| [Peak area/gTS] | GRF-tH1ura3 | pFlat3-ERG11 |
| Squalene | 9.93 | 5.46 |
| Lanosterol | 0.83 | 0.43 |
| Zymosterol | 1.18 | 1.64 |
| Ergosterol | 4.71 | 7.31 |
| Fecosterol | 1.10 | 1.32 |
| Episterol | 1.04 | 1.67 |
| Dimethylzymosterol | 0.34 | 0.53 |

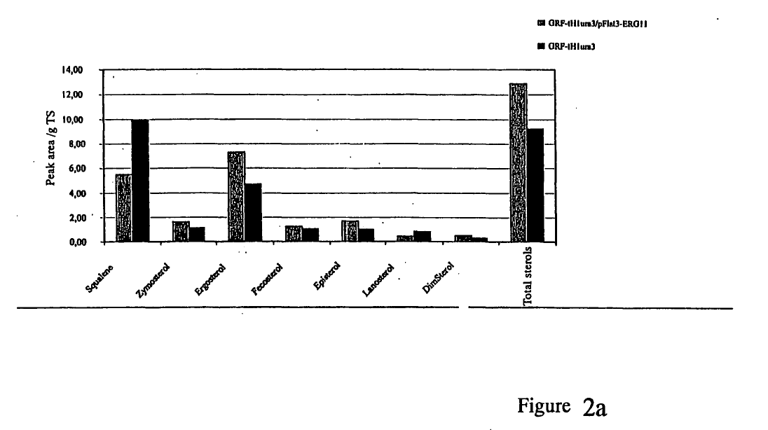

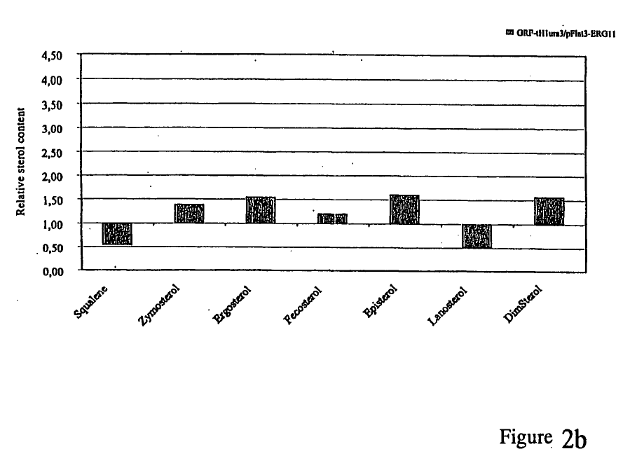

FIGS. 2a and 2b show the absolute increase (2a) and percent increase (2b) in the content of individual sterols in S. cerevisae GRF tH1ura3/pFlat1-ERG1 in comparison with the starting strain S. cerevisae GRF tH1ura3.

### Example 4

Combined Expression of ERG1 and ERG11 in S. cerevisiae GRF tH1ura3 (t-HMG+ERG1+ERG11)

The two episomal expression vectors pFlat1-ERG1 (see Example 2) and pFlat3-ERG11 (see Example 3) were transformed together and simultaneously into the yeast strain S. cerevisiae GRF tH1ura3, and the two genes ERG1 and ERG11 were expressed simultaneously under the control of the ADH promoter and the tryptophan terminator.

The yeast strain S. cerevisiae GRF-tH1ura3/pFlat1-ERG1/pFlat3-ERG11 thus obtained was then cultured in a culture volume of 20 ml in WMXIII medium at 28° C. and 160 rpm for 48 hours. Subsequently, 500 μl of this preculture were transferred to a 50 ml main culture of the same medium and cultured in a baffled flask at 28° C. and 160 rpm for 4 days.

After 4 days, the sterols were extracted according to Example 1 and analyzed by means of gas chromatography, resulting in the values listed in Table 4. The percentages are based on the yeast dry weight.

TABLE 4

| Sterol content | S. cerevisiae | S. cerevisiae GRF-tH1ura3 |
|---|---|---|
| [Peak area/gTS] | GRF-tH1ura3 | pFlat1-ERG1/pFlat3-ERG11 |
| Squalene | 9.93 | 9.81 |
| Lanosterol | 0.83 | 3.43 |
| Zymosterol | 1.18 | 3.42 |
| Ergosterol | 4.71 | 8.15 |
| Fecosterol | 1.10 | 2.22 |
| Episterol | 1.04 | 1.91 |
| Dimethylzymosterol | 0.34 | 0.75 |

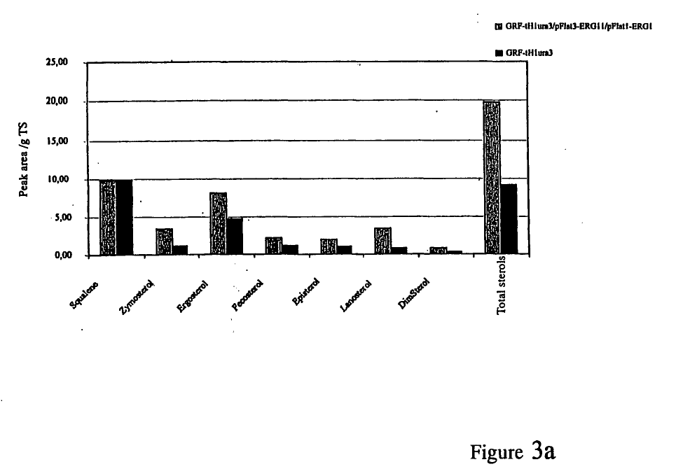

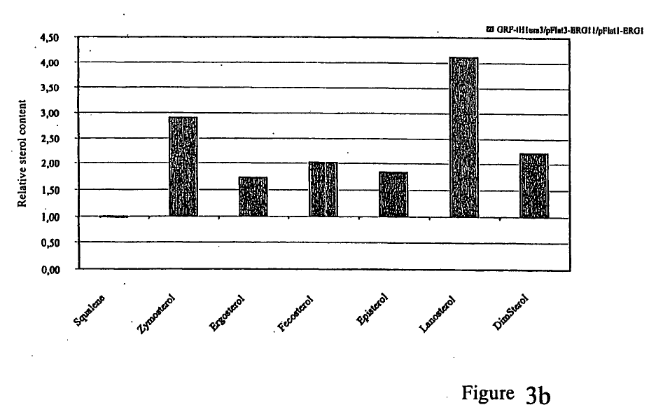

FIGS. 3a and 3b show the absolute increase (3a) and percent increase (3b) in the content of individual sterols in S. cerevisiae GRF tH1ura3/pFlat1-ERG1/pFlat3-ERG11 in comparison with the starting strain S. cerevisiae GRF tH1ura3.

Since it is known from the state of the art (Tainaka et al., J, Ferment. Bioeng. 1995, 79, 64-66) that overexpressin of ERG11 does not substantially increase the ergosterol content in yeast and since Example 1 shows that expression of a t-HMG does not substantially increase the ergosterol content in yeast, the increase in the ergosterol content by 100% as a result of overexpression of both genes must be referred to as surprising.

We claim:

1. A method for preparing zymosterol and/or the biosynthetic intermediates and/or secondary products thereof by culturing organisms which, compared to the wild type, have increased lanosterol-C14-demethylase activity and increased HMG-CoA-reductase activity.

2. A method as claimed in claim 1, wherein the lanosterol-C14-demethylase activity is increased by increasing the gene expression of a nucleic acid encoding a lanosterol C14-demethylase compared with the wild type.

3. A method as claimed in claim 2, wherein gene expression is increased by introducing into the organism one or more nucleic acids encoding a lanosterol C14-demethylase.

4. A method as claimed in claim 3, wherein nucleic acids are introduced, which encode proteins comprising the amino acid sequence SEQ. ID. NO. 2 or a sequence which is derived from this sequence by substitution, insertion or deletion of amino acids and which is at least 30% identical at the amino acid level to the sequence SEQ. ID. NO. 2, which proteins have the enzymic property of a lanosterol C14-demethylase.

5. A method as claimed in claim 4, wherein a nucleic acid comprising the sequence SEQ. ID. NO. 1 is introduced.

6. A method as claimed in any of claims 1 to 5, wherein the HMG-CoA-reductase activity is increased by increasing the gene expression of a nucleic acid encoding an HMG-CoA reductase compared with the wild type.

7. A method as claimed in claim 6, wherein gene expression is increased by introducing into the organism a nucleic acid construct comprising a nucleic acid encoding an HMG-CoA reductase whose expression in said organism is subject to a reduced regulation, in comparison with the wild type.

8. A method as claimed in claim 7, wherein the nucleic acid construct comprises a promoter which is subject in said organism to a reduced regulation, in comparison with the wild-type promoter.

9. A method as claimed in claim 7 or 8, wherein the HMG-CoA-reductase-encoding nucleic acid used is a nucleic acid whose expression in said organism is subject to a reduced regulation, in comparison with the orthologous nucleic acid intrinsic to said organism.

10. A method as claimed in claim 9, wherein the HMG-CoA-reductase-encoding nucleic acid used is a nucleic acid which encodes only the catalytic region of said HMG-CoA reductase.

11. A method as claimed in claim 10, wherein nucleic acids are introduced, which encode proteins comprising the amino acid sequence SEQ. ID. NO. 4 or a sequence which is derived from this sequence by substitution, insertion or deletion of amino acids and which is at least 30% identical at the amino acid level to the sequence SEQ. ID. NO. 4, which proteins have the enzymic property of a HMG-CoA reductase.

12. A method as claimed in claim 11, wherein a nucleic acid comprising the sequence SEQ. ID. NO. 3 is introduced.

13. A method as claimed in any of claims 1 to 12, wherein an organism is used which, compared to the wild type, additionally has an increased squalene-epoxidase activity.

14. A method as claimed in claim 13, wherein the squalene-epoxidase activity is increased by increasing the gene expression of a nucleic acid encoding a squalene epoxidase compared with the wild type.

15. A method as claimed in claim 14, wherein gene expression is increased by introducing into the organism one or more nucleic acids encoding a squalene epoxidase.

16. A method as claimed in claim 15, wherein nucleic acids are introduced, which encode proteins comprising the amino acid sequence SEQ. ID. NO. 6 or a sequence which is derived from this sequence by substitution, insertion or deletion of amino acids and which is at least 30% identical at the amino acid level to the sequence SEQ. ID. NO. 6, which proteins have the enzymic property of a squalene epoxidase.

17. A method as claimed in claim 16, wherein a nucleic acid comprising the sequence SEQ. ID. NO. 5 is introduced.

18. A method as claimed in any of claims 1 to 17, wherein the organism used is yeast.

19. A method as claimed in any of claims 1 to 18, wherein, after culturing, the organism is harvested and then zymosterol and/or its biosynthetic intermediates and/or secondary products are isolated from said organism.

20. A nucleic acid construct, comprising nucleic acids encoding a lanosterol C14-demethylase and nucleic acids encoding an HMG-CoA reductase, which are functionally linked to one or more regulatory signals which ensure transcription and translation in organisms.

21. A nucleic acid construct as claimed in claim 20, additionally comprising nucleic acids encoding a squalene epoxidase.

22. A combination of nucleic acid constructs, which comprises
a) a first nucleic acid construct comprising nucleic acids encoding a lanosterol C14-demethylase, which are functionally linked to one or more regulatory signals which ensure transcription and translation in organisms and
b) a second nucleic acid construct comprising nucleic acids encoding an HMG-CoA reductase, which are functionally linked to one or more regulatory signals which ensure transcription and translation in organisms.

23. A combination as claimed in claim 22, which comprises
c) yet another, third nucleic acid construct comprising nucleic acids encoding a squalene epoxidase, which are functionally linked to one or more regulatory signals which ensure transcription and translation in organisms.

24. A nucleic acid construct or combination of nucleic acid constructs as claimed in any of claims 20 to 23, wherein the regulatory signals comprise one or more promoters and one or more terminators, which ensure transcription and translation in organisms.

25. A nucleic acid construct or combination of nucleic acid constructs as claimed in claim 24, wherein regulatory signals are used, which ensure transcription and translation in yeasts.

26. A genetically modified organism, wherein the genetic modification increases the activity of a lanosterol C14-demethylase and an HMG-CoA reductase compared to a wild type.

27. A genetically modified organism as claimed in claim 26, wherein the increase in lanosterol C14-demethylase activity is caused by an increase in the gene expression of a nucleic acid encoding a lanosterol C14-demethylase, compared to the wild type.

28. A genetically modified organism as claimed in claim 27, which comprises two or more nucleic acids encoding a lanosterol C14-demethylase.

29. A genetically modified organism as claimed in any of claims 26 to 28, wherein the increase in HMG-CoA-reductase activity is caused by an increase in the gene expression of a nucleic acid encoding an HMG-CoA reductase, compared to the wild type.

30. A genetically modified organism as claimed in claim 29, which comprises a nucleic acid construct comprising a nucleic acid encoding an HMG-CoA reductase whose expression in said organism is subject to a reduced regulation, in comparison with the wild type.

31. A genetically modified organism as claimed in claim 30, wherein the nucleic acid construct comprises a promoter which is subject in said organism to a reduced regulation, in comparison with the wild type.

32. A genetically modified organism as claimed in claim 30 or 31, wherein the HMG-CoA-reductase-encoding nucleic acid used is a nucleic acid which encodes only the catalytic region of said HMG-CoA reductase.

33. A genetically modified organism as claimed in any of claims 26 to 32, wherein the genetic modification additionally increases the squalene-epoxidase activity compared to a wild type.

34. A genetically modified organism as claimed in claim 33, wherein the increase in squalene-epoxidase activity is caused by an increase in the gene expression of a nucleic acid encoding a squalene epoxidase, compared to the wild type.

35. A genetically modified organism as claimed in claim 34, which comprises two or more nucleic acids encoding a squalene-epoxidase activity.

36. A genetically modified organism as claimed in any of claims 26 to 35, which has, compared to the wild type, an increased content of zymosterol and/or of the biosynthetic intermediates and/or secondary products thereof.

37. A genetically modified organism as claimed in any of claims 26 to 36, wherein the organism used is yeast.

38. The use of a genetically modified organism as claimed in any of claims 26 to 37 for producing zymosterol and/or the biosynthetic intermediates and/or secondary products thereof.

39. A method for preparing genetically modified organisms as claimed in any of claims 26 to 37, wherein nucleic acids as claimed in any of claims 3 to 5 and nucleic acid constructs as claimed in any of claims 7 to 11 are introduced into the genome of the starting organism.

40. A method as claimed in claim 39, wherein additionally nucleic acids as claimed in any of claims 15 to 17 are introduced into the genome of the starting organism.

41. The use of the nucleic acids as claimed in any of claims 3 to 5 or 15 to 17 or of the nucleic acid constructs as claimed in any of claims 7 to 11 for increasing the content of zymosterol and/or the biosynthetic intermediates and/or secondary products thereof in organisms.


SEQUENCE LISTING (12 sequences)

SEQ ID NO: 1 - DNA, 1593 bp, Saccharomyces cerevisiae
  CDS (1)..(1593)
atgtctgctaccaagtcaatcgttggagaggcattggaatacgtaaacattggtttaagtcatttcttggctttaccattggcccaaagaatctctttgatcataataattcctttcatttacaatattgtatggcaattactatattctttgagaaaggaccgtccacctctagtgttttactggattccatgggtcggtagtgctgttgtgtacggtatgaagccatacgagtttttcgaagaatgtcaaaagaaatacggtgatattttttcattcgttttgttaggaagagtcatgactgtgtatttaggaccaaagggtcacgaatttgtcttcaacgctaagttggcagatgtttcagcagaagctgcttacgctcatttgactactccagttttcggtaaaggtgttatttacgattgtccaaattctagattgatggagcaaaagaagtttgttaagggtgctctaaccaaagaagccttcaagagctacgttccattgattgctgaagaagtgtacaagtacttcagagactccaaaaacttccgtttgaatgaaagaactactggtactattgacgtgatggttactcaacctgaaatgactattttcaccgcttcaagatcattattgggtaaggaaatgagagcaaaattggataccgattttgcttacttgtacagtgatttggataagggtttcactccaatcaacttcgtcttccctaacttaccattggaacactatagaaagagagatcacgctcaaaaggctatctccggtacttacatgtctttgattaaggaaagaagaaagaacaacgacattcaagacagagatttgatcgattccttgatgaagaactctacctacaaggatggtgtgaagatgactgatcaagaaatcgctaacttgttaattggtgtcttaatgggtggtcaacatacttctgctgccacttctgcttggattttgttgcacttggctgaaagaccagatgtccaacaagaattgtacgaagaacaaatgcgtgttttggatggtggtaagaaggaattgacctacgatttattacaagaaatgccattgttgaaccaaactattaaggaaactctaagaatgcaccatccattgcactctttgttccgtaaggttatgaaagatatgcacgttccaaacacttcttatgtcatcccagcaggttatcacgttttggtttctccaggttacactcatttaagagacgaatacttccctaatgctcaccaattcaacattcaccgttggaacaaagattctgcctcctcttattccgtcggtgaagaagtcgattacggtttcggtgccatttctaagggtgtcagctctccatacttacctttcggtggtggtagacacagatgtatcggtgaacactttgcttactgtcagctaggtgttctaatgtccatttttatcagaacattaaaatggcattacccagagggtaagaccgttccacctcctgactttacatctatggttactcttccaaccggtccagccaagatcatctgggaaaagagaaatccagaacaaaagatctaa

SEQ ID NO: 2 - protein, 530 aa, Saccharomyces cerevisiae
MSATKSIVGEALEYVNIGLSHFLALPLAQRISLIIIIPFIYNIVWQLLYSLRKDRPPLVFYWIPWVGSAVVYGMKPYEFFEECQKKYGDIFSFVLLGRVMTVYLGPKGHEFVFNAKLADVSAEAAYAHLTTPVFGKGVIYDCPNSRLMEQKKFVKGALTKEAFKSYVPLIAEEVYKYFRDSKNFRLNERTTGTIDVMVTQPEMTIFTASRSLLGKEMRAKLDTDFAYLYSDLDKGFTPINFVFPNLPLEHYRKRDHAQKAISGTYMSLIKERRKNNDIQDRDLIDSLMKNSTYKDGVKMTDQEIANLLIGVLMGGQHTSAATSAWILLHLAERPDVQQELYEEQMRVLDGGKKELTYDLLQEMPLLNQTIKETLRMHHPLHSLFRKVMKDMHVPNTSYVIPAGYHVLVSPGYTHLRDEYFPNAHQFNIHRWNKDSASSYSVGEEVDYGFGAISKGVSSPYLPFGGGRHRCIGEHFAYCQLGVLMSIFIRTLKWHYPEGKTVPPPDFTSMVTLPTGPAKIIWEKRNPEQKI

SEQ ID NO: 3 - DNA, 1578 bp, Artificial Sequence
  Description of Artificial Sequence Truncated HMG construct
atggaccaattggtgaaaactgaagtcaccaagaagtcttttactgctcctgtacaaaaggcttctacaccagttttaaccaataaaacagtcatttctggatcgaaagtcaaaagtttatcatctgcgcaatcgagctcatcaggaccttcatcatctagtgaggaagatgattcccgcgatattgaaagcttggataagaaaatacgtcctttagaagaattagaagcattattaagtagtggaaatacaaaacaattgaagaacaaagaggtcgctgccttggttattcacggtaagttacctttgtacgctttggagaaaaaattaggtgatactacgagagcggttgcggtacgtaggaaggctctttcaattttggcagaagctcctgtattagcatctgatcgtttaccatataaaaattatgactacgaccgcgtatttggcgcttgttgtgaaaatgttataggttacatgcctttgcccgttggtgttataggccccttggttatcgatggtacatcttatcatataccaatggcaactacagagggttgtttggtagcttctgccatgcgtggctgtaaggcaatcaatgctggcggtggtgcaacaactgttttaactaaggatggtatgacaagaggcccagtagtccgtttcccaactttgaaaagatctggtgcctgtaagatatggttagactcagaagagggacaaaacgcaattaaaaaagcttttaactctacatcaagatttgcacgtctgcaacatattcaaacttgtctagcaggagatttactcttcatgagatttagaacaactactggtgacgcaatgggtatgaatatgatttctaaaggtgtcgaatactcattaaagcaaatggtagaagagtatggctgggaagatatggaggttgtctccgtttctggtaactactgtaccgacaaaaaaccagctgccatcaactggatcgaaggtcgtggtaagagtgtcgtcgcagaagctactattcctggtgatgttgtcagaaaagtgttaaaaagtgatgtttccgcattggttgagttgaacattgctaagaatttggttggatctgcaatggctgggtctgttggtggatttaacgcacatgcagctaatttagtgacagctgttttcttggcattaggacaagatcctgcacaaaatgttgaaagttccaactgtataacattgatgaaagaagtggacggtgatttgagaatttccgtatccatgccatccatcgaagtaggtaccatcggtggtggtactgttctagaaccacaaggtgccatgttggacttattaggtgtaagaggcccgcatgctaccgctcctggtaccaacgcacgtcaattagcaagaatagttgcctgtgccgtcttggcaggtgaattatccttatgtgctgccctagcagccggccatttggttcaaagtcatatgacccacaacaggaaacctgctgaaccaacaaaacctaacaatttggacgccactgatataaatcgtttgaaagatgggtccgtcacctgcattaaatcctaa

SEQ ID NO: 4 - protein, 525 aa, Artificial Sequence
  Description of Artificial Sequence Synthetic protein construct
MDQLVKTEVTKKSFTAPVQKASTPVLTNKTVISGSKVKSLSSAQSSSSGPSSSSEEDDSRDIESLDKKIRPLEELEALLSSGNTKQLKNKEVAALVIHGKLPLYALEKKLGDTTRAVAVRRKALSILAEAPVLASDRLPYKNYDYDRVFGACCENVIGYMPLPVGVIGPLVIDGTSYHIPMATTEGCLVASAMRGCKAINAGGGATTVLTKDGMTRGPVVRFPTLKRSGACKIWLDSEEGQNAIKKAFNSTSRFARLQHIQTCLAGDLLFMRFRTTTGDAMGMNMISKGVEYSLKQMVEEYGWEDMEVVSVSGNYCTDKKPAAINWIEGRGKSVVAEATIPGDVVRKVLKSDVSALVELNIAKNLVGSAMAGSVGGFNAHAANLVTAVFLALGQDPAQNVESSNCITLMKEVDGDLRISVSMPSIEVGTIGGGTVLEPQGAMLDLLGVRGPHATAPGTNARQLARIVACAVLAGELSLCAALAAGHLVQSHMTHNRKPAEPTKPNNLDATDINRLKDGSVTCIKS

SEQ ID NO: 5 - DNA, 1491 bp, Saccharomyces cerevisiae
  CDS (1)..(1491)
atgtctgctgttaacgttgcacctgaattgattaatgccgacaacacaattacctacgatgcgattgtcatcggtgctggtgttatcggtccatgtgttgctactggtctagcaagaaagggtaagaaagttcttatcgtagaacgtgactgggctatgcctgatagaattgttggtgaattgatgcaaccaggtggtgttagagcattgagaagtctgggtatgattcaatctatcaacaacatcgaagcatatcctgttaccggttataccgtctttttcaacggcgaacaagttgatattccatacccttacaaggccgatatccctaaagttgaaaaattgaaggacttggtcaaagatggtaatgacaaggtcttggaagacagcactattcacatcaaggattacgaagatgatgaaagagaaaggggtgttgcttttgttcatggtagattcttgaacaacttgagaaacattactgctcaagagccaaatgttactagagtgcaaggtaactgtattgagatattgaaggatgaaaagaatgaggttgttggtgccaaggttgacattgatggccgtggcaaggtggaattcaaagcccacttgacatttatctgtgacggtatcttttcacgtttcagaaaggaattgcacccagaccatgttccaactgtcggttcttcgtttgtcggtatgtctttgttcaatgctaagaatcctgctcctatgcacggtcacgttattcttggtagtgatcatatgccaatcttggtttaccaaatcagtccagaagaaacaagaatcctttgtgcttacaactctccaaaggtcccagctgatatcaagagttggatgattaaggatgtccaacctttcattccaaagagtctacgtccttcatttgatgaagccgtcagccaaggtaaatttagagctatgccaaactcctacttgccagctagacaaaacgacgtcactggtatgtgtgttatcggtgacgctctaaatatgagacatccattgactggtggtggtatgactgtcggtttgcatgatgttgtcttgttgattaagaaaataggtgacctagacttcagcgaccgtgaaaaggttttggatgaattactagactaccatttcgaaagaaagagttacgattccgttattaacgttttgtcagtggctttgtattctttgttcgctgctgacagcgataacttgaaggcattacaaaaaggttgtttcaaatatttccaaagaggtggcgattgtgtcaacaaacccgttgaatttctgtctggtgtcttgccaaagcctttgcaattgaccagggttttcttcgctgtcgctttttacaccatttacttgaacatggaagaacgtggtttcttgggattaccaatggctttattggaaggtattatgattttgatcacagctattagagtattcaccccatttttgtttggtgagttgattggttaa

SEQ ID NO: 6 - protein, 496 aa, Saccharomyces cerevisiae
MSAVNVAPELINADNTITYDAIVIGAGVIGPCVATGLARKGKKVLIVERDWAMPDRIVGELMQPGGVRALRSLGMIQSINNIEAYPVTGYTVFFNGEQVDIPYPYKADIPKVEKLKDLVKDGNDKVLEDSTIHIKDYEDDERERGVAFVHGRFLNNLRNITAQEPNVTRVQGNCIEILKDEKNEVVGAKVDIDGRGKVEFKAHLTFICDGIFSRFRKELHPDHVPTVGSSFVGMSLFNAKNPAPMHGHVILGSDHMPILVYQISPEETRILCAYNSPKVPADIKSWMIKDVQPFIPKSLRPSFDEAVSQGKFRAMPNSYLPARQNDVTGMCVIGDALNMRHPLTGGGMTVGLHDVVLLIKKIGDLDFSDREKVLDELLDYHFERKSYDSVINVLSVALYSLFAADSDNLKALQKGCFKYFQRGGDCVNKPVEFLSGVLPKPLQLTRVFFAVAFYTIYLNMEERGFLGLPMALLEGIMILITAIRVFTPFLFGELIG

SEQ ID NO: 7 - DNA, 35 bp, Artificial Sequence
  Description of Artificial Sequence Synthetic primer
ctgcggccgcatcatggaccaattggtgaaaactg

SEQ ID NO: 8 - DNA, 32 bp, Artificial Sequence
  Description of Artificial Sequence Synthetic primer
aactcgagagacacatggtgctgttgtgcttc

SEQ ID NO: 9 - DNA, 33 bp, Artificial Sequence
  Description of Artificial Sequence Synthetic primer
ctgcggccgcatcatgtctgctgttaacgttgc

SEQ ID NO: 10 - DNA, 30 bp, Artificial Sequence
  Description of Artificial Sequence Synthetic primer
ttctcgagttaaccaatcaactcaccaaac

SEQ ID NO: 11 - DNA, 35 bp, Artificial Sequence
  Description of Artificial Sequence Synthetic primer
ctgcggccgcaggatgtctgctaccaagtcaatcg

SEQ ID NO: 12 - DNA, 34 bp, Artificial Sequence
  Description of Artificial Sequence Synthetic primer
atctcgagcttagatcttttgttctggatttctc